(19) United States
(12) Patent Application Publication
(10) Pub. No.: US 2024/0280571 A1
(43) Pub. Date: Aug. 22, 2024

(54) POROUS SENSORS, METHODS OF MAKING, AND METHOD OF USING

(71) Applicant: VANDERBILT UNIVERSITY, Nashville, TN (US)

(72) Inventors: Rabeb Layouni, Nashville, TN (US); Paul E. Laibinis, Nashville, TN (US); Sharon M. Weiss, Nashville, TN (US)

(21) Appl. No.: 18/422,496

(22) Filed: Jan. 25, 2024

Related U.S. Application Data
(60) Provisional application No. 63/481,688, filed on Jan. 26, 2023.

(51) Int. Cl. G01N 33/543; G01N 21/78

(52) U.S. Cl. CPC G01N 33/54386; G01N 21/78

(57) ABSTRACT

The present disclosure provides for porous membrane sensors, methods of making porous membrane sensors, methods of using porous membrane sensors, and the like. The present disclosure provides for reliable rapid diagnostic tests (RDT) that are low-cost, are widely deployable, are highly sensitive, and can provide results quickly.

### CROSS-REFERENCE TO RELATED APPLICATIONS

This application claims priority to U.S. provisional application entitled “Porous Silicon on Paper Optical Sensor, Methods of Making and Methods of Using” having Ser. No. 63/481,688 filed on Jan. 26, 2023, which is entirely incorporated herein by reference.

### FEDERAL SPONSORSHIP

This invention was made with government support under Grant No. ECCS2037673, awarded by U.S. National Science Foundation. The government has certain rights in the invention.

### BACKGROUND

Reliable rapid diagnostic test (RDT) for infections that is cost effective, sensitive and provides results in 30 min or less is desirable to reduce the spread of infection. The most common form of RDT is a paper-based immunoassay with a color change readout, but quantification of the color change is challenging and achieving sufficient sensitivity to detect diseases at an early stage is difficult. Thus, there is a need to address these problems.

### SUMMARY

Embodiments of the present disclosure provide for porous membrane sensors, methods of making porous membrane sensors, methods of using porous membrane sensors, and the like.

The present disclosure provides for a sensor, comprising: optionally, a top cover having at least one opening through the top cover, wherein the top cover has a first side and a second side opposite the first side; a porous membrane, optionally, disposed on a first side of a membrane support, wherein the porous membrane has a first side and a second side opposite the first side of the porous support, wherein the porous membrane includes a plurality of pores that extend from the first side of the porous membrane to the second side of the porous membrane, wherein the membrane support has a second side opposite the first side of the membrane support, wherein the second side of the top cover is disposed on the first side of the membrane support; an absorbent layer having a first side and a second side opposite the first side, wherein the second side of the membrane support is disposed on the first side of the absorbent layer, wherein the absorbent layer has the characteristic of being able to absorb a fluid; and, optionally, a bottom cover having a first side and a second side opposite the first side, wherein the second side of the absorbent layer is disposed on the first side of the bottom cover. In an aspect, the present disclosure includes the top cover, the membrane support, and the bottom cover.

The present disclosure provides for a sensor having: a porous membrane disposed on a first side of a membrane support (optional), wherein the porous membrane has a first side and a second side opposite the first side of the porous support, wherein the porous membrane includes a plurality of pores that extend from the first side of the porous membrane to the second side of the porous membrane, wherein the membrane support has a second side opposite the first side of the membrane support; and an absorbent layer having a first side and a second side opposite the first side, wherein the second side of the membrane support is disposed on the first side of the absorbent layer, wherein the absorbent layer has the characteristic of being able to absorb a fluid.

The present disclosure provides for a method of detecting a target, comprising: exposing a sample fluid on the porous membrane of the sensor as described above or herein, wherein if the sample fluid includes the target, the target will be associated with the binding agent (e.g., the binding agent is bonded to the surface of the porous membrane directly or indirectly) and produce a color change in the porous membrane, wherein if the sample fluid does not include the target, the color change does not occur; and determining if a color change occurs, and if a color change occurs, the target is present in the sample fluid. In an aspect, determining can include quantification of the amount of the target in the fluid sample.

### BRIEF DESCRIPTION OF DRAWINGS

Further aspects of the present disclosure will be more readily appreciated upon review of the detailed description of its various embodiments, described below, when taken in conjunction with the accompanying drawings.

FIG. 1.1 illustrates PSi-on-paper for detection of BSA adsorption. FIG. 1(a) is a photo taken after 20 μL of 150 μM BSA solution was drop cast on the PSi sample window. FIG. 1(b) is a photo taken after the BSA solution passed through PSi (no droplet remains on the surface). FIG. 1(c) is a plot illustrating Fast Fourier Transform processed reflectance measurements before and after the BSA exposure. The plot shows an increase in the PSi membrane optical thickness, which indicates attachment of BSA proteins in the pores.

FIG. 2.1(a) illustrates a schematic showing PSi membrane on paper with inset illustrating the capture of biomolecules in the pores. FIG. 2.1(b) is a photo of PSi on paper rapid diagnostic test (RDT).

FIG. 2.2 illustrates real-time response of biotin-functionalized PSi on paper sensor upon exposure to streptavidin target molecules and water washes: (FIG. 2.2(a)) without and (FIG. 2.2(b)) with the use of externally assisted drying to reduce the response time. Each solution introduced to the sensor had a volume of 10 μL.

FIG. 3.1(a) illustrates a schematic illustration of PSi in flow-over and flow-through schemes. The flow-through scheme achieves a faster response time for streptavidin detection. FIG. 3.1(b) illustrates snapshots of velocity and concentration distributions in PSi in flow-through scheme, calculated using COMSOL. (Reproduced with permission from Ref. 10 in Example 2).

FIG. 3.2(a) illustrates a schematic of PSi smartphone biosensor. FIG. 3.2(b) illustrates a top-view SEM image of PSi microcavity. FIG. 3.2(c) illustrates a typical reflectance spectra of PSi microcavity. Blue and red curves are reflectance spectra before and after adding molecules, respectively. The red shaded region indicates the spectral bandwidth of the filter in front of the image sensor in the smartphone. Molecular attachment leads to a reduced light intensity measured by the smartphone.

FIG. 3.3 illustrates schematic illustration of paper-based PSi diagnostic test.

FIG. 3.4 illustrates porous silicon on paper test design: PSi free-standing membrane sits on nitrocellulose (NC) membrane. An absorbent pad is inserted under the NC membrane and a bottom and top wax-printed layers are used to seal all the other layers into on paper test.

FIG. 3.5 illustrates scanning electron microscope (SEM) images of PSi membrane. FIG. 3.5(a) illustrates a top view SEM image of PSi free-standing membrane with 33 nm average pore size. FIG. 3.5(b) illustrates a cross-sectional SEM image of PSi free-standing membrane with a thickness of 15.5 μm.

FIG. 3.6 illustrates ATR-FTIR absorbance spectrum of PSi free-standing membrane oxidized at 500° C. for 5 minutes compared to unoxidized PSi.

FIG. 3.7(a-i) PSi-on-paper fabrication process: (a) PSi film formation by electrochemical etching; (b) PSi membrane lift-off from silicon substrate; (c) oxidation of PSi membrane at 500° C. in air; (d) placement of nitrocellulose (NC) membrane at bottom of Petri dish filled with water; (c) transfer of PSi membrane to NC membrane in water by slowly removing the water with a pipettor; (f) drying the PSi-NC membrane system in air at room temperature; (g) assembly of PSi/NC and top wax paper layer; (h) removal of the rest of the NC membrane; (i) attachment of the absorbent pad and bottom wax paper layer.

FIG. 3.8 illustrates one embodiment of an assembled PSi on paper test device.

FIG. 3.9 illustrates simulated and measured optical properties of one design of a PSi-on-paper test strip. Reflectance spectra and characteristic optical thickness plots are shown.

FIG. 3.10 illustrates modification of an oxidized free-standing PSi membrane with APTES and biotin. Corresponding ATR-FTIR spectra are shown.

FIG. 3.11 illustrates the optical thickness change upon exposure of biotin-functionalized PSi on paper test to streptavidin.

FIG. 3.12 illustrates a comparison of saturation time for porous silicon film thickness of 50 μm and 20 μm at 80 μM, 10 μM, 5 μM and 1 μM, shows a faster sensor response for the thinner membrane.

FIG. 4.1A illustrates a cross-sectional view of an embodiment of a sensor, while FIG. 4.1B illustrates a perspective view of sensor. FIG. 4.1C illustrates a sample fluid disposed on the porous membrane through the opening of the top cover.

FIG. 4.2A illustrates a cross-sectional view of another embodiment of a sensor, which is a lateral flow configuration as shown in FIG. 4.2B.

### DETAILED DESCRIPTION

In general, the present disclosure provides for porous sensors (e.g., porous silicon-based sensors), method of making porous sensors (e.g., porous silicon sensors), methods of using porous sensors (e.g., porous silicon sensors) and the like. Additional details are provided herein and in the Examples.

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, inorganic chemistry, synthetic chemistry, and the like, which are within the skill of the art. Such techniques are explained fully in the literature.

The following description and examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is in bar or psig. Standard temperature and pressure are defined as 25° C. and 1 bar.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible. Different stereochemistry is also possible, such as products of cis or trans orientation around a carbon-carbon double bond or syn or anti addition could be both possible even if only one is drawn in an embodiment.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

### Definitions

By “chemically feasible” is meant a bonding arrangement or a compound where the generally understood rules of organic structure are not violated. The structures disclosed herein, in all of their embodiments are intended to include only “chemically feasible” structures, and any recited structures that are not chemically feasible, for example in a structure shown with variable atoms or groups, are not intended to be disclosed or claimed herein. However, if a bond appears to be intended and needs the removal of a group such as a hydrogen from a carbon, the one of skill would understand that a hydrogen could be removed to form the desired bond.

The term “detectable” refers to the ability to detect a signal over the background signal. “Detectable” can include post-processing (e.g., Fourier-transform, artificial intelligence, and the like) of data so that the signal over the background signal.

The term “detectable signal” is a signal derived from a color change upon the interaction of a binding agent and a target. The color change is a structural color change. Structural color is color that is produced, at least in part, by microscopically structured surfaces that interfere with visible light contacting the surface. The structural color is color caused by physical phenomena including the scattering, refraction, reflection, interference, and/or diffraction of light, unlike color caused by the absorption or emission of visible light through coloring matters (e.g., dyes, pigments, etc.). The detectable signal (e.g., color change) is detectable and distinguishable from other background signals (e.g., colors). In other words, there is a measurable and statistically significant difference (e.g., a statistically significant difference is enough of a difference to distinguish among the detectable signal and the background, such as about 0.1%, 1%, 3%, 5%, 10%, 15%, 20%, 25%, 30%, 40% or more difference between the detectable signal and the background) between the detectable signal and the background. Standards and/or calibration curves can be used to determine the relative intensity of the detectable signal and/or the background.

### General Discussion

The present disclosure provides for porous membrane sensors, methods of making porous membrane sensors, methods of using porous membrane sensors, and the like. The present disclosure provides for reliable rapid diagnostic tests (RDT) that are low-cost, widely deployable, highly sensitive, and can provide results quickly (e.g., in less than 60 min or less than 30 minutes).

In an aspect, the present disclosure provides for optical sensing using a porous membrane sensor that can include a porous membrane (e.g., porous silicon (PSi)) and membrane support (e.g., paper), as a highly sensitive, quantitative, and reliable platform for rapid diagnostic tests (RDTs). In an aspect, the porous membrane sensor can include PSi free-standing membrane that sits on the membrane support (e.g., nitrocellulose membrane support), which in turn is contacted with an absorbent layer below. The analyte sample flows through the PSi film and membrane support to the absorbent layer; capillary forces drive the analyte flow. When the analyte (e.g., target) is present in the PSi film, the effective refractive index of the film increases, and the characteristic Fabry-Perot reflectance fringes shift to longer wavelengths, produce a detectable signal (e.g., color change), and can be detected and measured. Optical measurements can be carried out with a spectrometer (e.g., a portable spectrometer in an aspect) or smartphone. For example, a porous membrane sensor for biosensing on a biotin-streptavidin protein assay PSi on paper test was performed and yielded a spectral shift with a spectrometer, indicating capture of analyte molecules.

The present disclosure provides various advantages, one of which is the large active sensing surface area of the porous membrane. Another advantage is that the pore diameters can be controlled so that the pore diameters are large as compared to the size of the binding agent and target molecules. Improved mass transport of the porous membrane sensors of the present disclosure enables a “flow-through” configuration that overcomes diffusion limitation and enhances sensor sensitivity and its applicability to point of care rapid diagnostics. Thus, the present disclosure provides for porous membrane sensors that are capable of rapid, accurate, quantitative, and high sensitivity detection of targets that can significantly advance the capabilities of rapid diagnostic testing.

In an aspect, the present disclosure provides for a sensor that can detect the presence of a target (e.g., a virus, contaminant (e.g., heavy metal), and the like) in a fluid sample (e.g., saliva, water from a stream, and the like). The sensor includes a porous membrane and an absorbent layer. The porous membrane will be discussed in more detail below and herein. The porous membrane is exposed to the fluid sample, the fluid sample flows through the porous membrane and into the absorbent layer. The porous membrane includes pores that include binding agents attached or bonded (e.g., directly or indirectly) to the walls of the pores. The fluid sample is drawn through the porous membrane by capillary forces. If the fluid sample includes the target, the target can bond or otherwise interact with the binding agent to produce a color change (e.g., the porous membrane will change color to produce a detectable signal) that is detectable and measurable. If the fluid sample does not include the target then a color change is not produced (e.g., once the fluid drains out of the porous silicon). The color change is measure after the sample fluid flows out of the porous membrane (e.g., the target will remain in the porous membrane and a color change will result). The detection of color change can be done when the porous membrane is “dry” or “wet”, but the comparison to if a color change occurs should be done when the porous membrane is in the same state (e.g., dry or wet). For example, if a sample that does not include the target flow through the porous membrane, but the porous membrane is still “wet”, a color change may occur relative to the porous membrane in a “dry” state, which is not the appropriate comparison. In one example, the color of the porous membrane is measured in the dry state. Then after flowing the fluid sample through the porous membrane and allowing the porous membrane to return to the dry state, the color is measured again. If the target is present in the fluid sample and bound to the binding agent within the porous membrane, a color change will be present. In this example, the comparison before and after exposure to the fluid sample is performed in the dry state.

The sensor can include one or more absorbent supports. In an aspect, the sensor includes one absorbent support. The absorbent layer can be made of cellulose. In an aspect, the absorbent layer can include absorbent materials such as pulp fibers. cotton fibers, cellulose fibers, rayon fibers, hydrogels, sodium polyacrylate, hygroscopic materials, and the like. The absorbent support can have a thickness of about 0.5 to 5 millimeters or about 0.5 to 1.5 millimeters.

The sensor can also include a membrane support upon which the porous membrane is disposed. In other aspects, the porous membrane can be of a thickness that a membrane support is not necessary. The membrane support can be made of a cellulose material, glass fibers, ceramic, and the like. In an aspect, the membrane support is made of paper. In an embodiment, the membrane support is made of nitrocellulose. The membrane support can have a thickness of about 0.5 to 1.5 millimeters.

The sensor can also include a top cover that includes an opening. The top cover is placed over the porous membrane. In an aspect, the porous membrane is disposed on the membrane support and the top cover is disposed on the porous membrane and the membrane support. In an embodiment, the opening of the top cover is aligned with the porous membrane so that the fluid sample can be disposed onto the porous membrane. In an embodiment, the opening of the top cover is not aligned with the porous membrane and a lateral flow system using a channel can flow the fluid sample onto the porous membrane. The top cover can have a thickness of about 0.5 to 1.5 millimeters. The opening of the top cover can be about 1 millimeter to 5 centimeters, about 10 millimeters to 5 centimeters or about 100 millimeters to 2 centimeters. The top cover can be made of material that can act as a flexible barrier and include an opening. In an aspect, the top cover can be made of hydrophobic material such as wax printed paper, plastic, rubber, silicone, or the like.

The sensor can also include a bottom cover. The bottom cover is disposed on the absorbent layer on the side opposite the porous membrane or the porous membrane/membrane support. The bottom cover can have a thickness of about 0.5 to 1.5 millimeters. The bottom cover can be made of hydrophobic material such as wax printed paper, plastic, rubber, silicone, or the like.

The length and width of top cover, porous membrane or membrane support, absorbent layer, and bottom cover can vary depending on the specifications of the sensor. In general, the length and width can be in the centimeter range (e.g., about 0.5 to 50 centimeters, about 1 to 20 centimeters or about 4 to 10 centimeters).

The top cover, porous membrane or membrane support, absorbent layer, and bottom cover can be connected together using an adhesive, tape (e.g., two-sided tape), or the like.

Having described the sensor generally, additional details are provided to describe the sensor, how the layers are positioned, and other features. In an aspect, the sensor includes a porous membrane and an absorbent layer, where the top cover, membrane support, and the bottom cover are optionally included.

In another aspect, the sensor includes a top cover, a porous membrane disposed on a membrane support, an absorbent layer, and a bottom cover. The top cover has at least one opening through the top cover. The top cover has a first side and a second side opposite the first side. The porous membrane is disposed on a first side of a membrane support. The porous membrane has a first side and a second side opposite the first side of the porous support. The porous membrane includes a plurality of pores that extend from the first side of the porous membrane to the second side of the porous membrane. The membrane support has a second side opposite the first side of the membrane support. The second side of the top cover is disposed on the first side of the membrane support. The absorbent layer has a first side and a second side opposite the first side. The second side of the membrane support is disposed on the first side of the absorbent layer. The absorbent layer has the characteristic of being able to absorb a fluid as described above and herein. The bottom cover has a first side and a second side opposite the first side. The second side of the absorbent layer is disposed on the first side of the bottom cover.

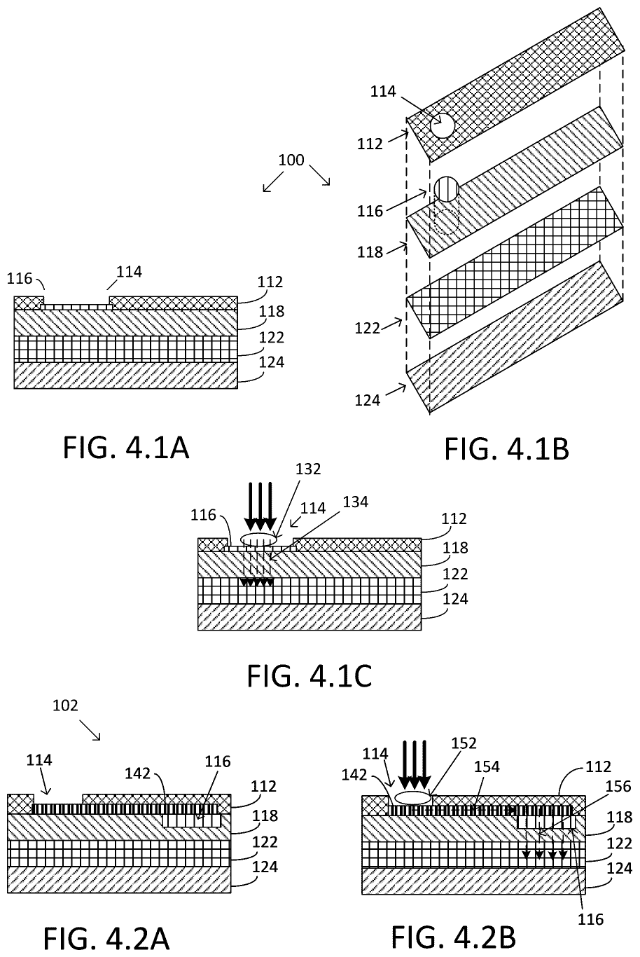

FIG. 4.1A and FIG. 4.2A illustrate two embodiments of the sensor. The various layers of the sensor are not drawn to scale but are illustrated merely to show the various layers. FIG. 4.1A illustrates a cross-sectional view of a sensor 100, while FIG. 4.1B illustrates a perspective view of sensor 100. Sensor 100 includes a top cover 112 including an opening 114. The top cover 112 is disposed on a first side of a membrane support 118 (which is optionally present). A porous membrane 116 is disposed on the membrane support 118. While the porous membrane 116 is disposed on the membrane support 118, the porous membrane 116 could be disposed in a recess of the membrane support 118 and/or the top cover 112. The porous membrane 116 is aligned with the opening 114 so that the fluid sample can be disposed on the porous membrane 116. The membrane support 118 is disposed on an absorbent layer 122 on the side opposite the top cover 112. A bottom cover 124 is disposed on the absorbent layer 122 on the side opposite the membrane support 118. It should be noted that while the top cover 112, membrane support 118, and bottom cover 124 are illustrated in FIG. 4.1A and FIG. 4.1B, each of these are optional.

FIG. 4.1C illustrates a sample fluid 132 disposed on the porous membrane 116 through the opening 114 of the top cover 112. The sample fluid 132 flows through the porous membrane 116 and the membrane support 118 and into the absorbent layer 122, where the sample fluid is absorbed.

FIG. 4.2A illustrates a cross-sectional view of a sensor 102, which is a lateral flow configuration as shown in FIG. 4.2B. Sensor 102 includes a top cover 112 including an opening 114. The top cover 112 is disposed on a first side of a membrane support 118. The membrane support 118 includes a porous membrane 116. While FIG. 4.2A illustrates the porous membrane 116 disposed in a recess of the membrane support 118, in another embodiment the porous membrane 116 is disposed on top of the membrane support 118, since the porous membrane 116 is very thin relative to the membrane support 118 and the top layer 112. The porous membrane 116 is not aligned with the opening 114. In an aspect, the top cover 112 is transparent or there is a transparent opening in the top cover 112 over the area directly above the porous membrane 116 so that the color change can be measured. The sensor 102 includes a pathway (e.g., a channel) 142 to flow 154 the sample fluid 152 to the porous membrane 116. The pathway 142 restricts flow of sample fluid from entering the membrane support 118 directly, instead requiring sample fluid to first flow through the porous membrane 116. The sample fluid 152 then flows 156 through the porous membrane 116. The membrane support 118 is disposed on an absorbent layer 122 on the side opposite the top cover 112. A bottom cover 124 is disposed on the absorbent layer 122 on the side opposite the membrane support 118. It should be noted that while the top cover 112, membrane support 118, and bottom cover 124 are illustrated in FIG. 4.2A and FIG. 4.2B, cach of these are optional.

The position of the pathway (e.g., channel) 142 in FIG. 4.2B is one configuration and other configuration options can include the pathway within the membrane support and the porous membrane positions towards the bottom of the membrane support near or adjacent the absorbent layer. Other configurations are also possible. The pathway 142 can be a conduit to flow the sample fluid and optionally include one or more filter materials (e.g., Whatman Grade 1 Chi chromatography paper, Whatman Grade 3 MM Chr chromatography paper, Whatman regenerated cellulose membrane 55, Whatman filter paper grade 50, Amershan Protran 0.45 nitrocellulose membrane, and the like) to remove component of the fluid sample. The removal of one or more components of the fluid sample may improve the detection (e.g., lower detection level, reduced false positives, and the like) of the sensor. The dimensions of the pathway are such that the sample fluid can flow to the porous membrane and expose the sample fluid to most of, if not all, of the porous membrane. The exact dimensions of the pathway will depend upon the various other components of the sensor can be designed to accomplish lateral flowing the sample fluid to the porous membrane.

In an embodiment, the porous membrane can be made of one or more types of materials such as ceramic, metal, metal oxide, polymer-based material, cellulose, carbon, porous metal organic framework, or fiberglass. In an aspect, the ceramic structure can be selected from silicon, silica, cordierite, alumina (e.g., γ-alumina, θ-alumina, δ-alumina), cordicrite-α-alumina, aluminosilicates, zirconia, germania, magnesia, titania, hafnia, silicon nitride, zircon mullite, spodumene, alumina-silica magnesia, zircon silicate, sillimanite, magnesium silicates, zircon, petalite, and combinations thereof. In an aspect, the porous membrane can be a porous silicon membrane, a porous alumina membrane, a porous silicon dioxide membrane, a porous germanium membrane, or a porous indium phosphide membrane. In an aspect, the porous membrane is a porous silicon membrane. In an embodiment, the porous membrane can include pores can extend through the porous membrane so that the sample fluid can flow through the porous membrane to the absorbent layer. In an embodiment, the pore of the porous membrane can be channels that extend through the entire thickness of the porous membrane. The channels can be straight, curvy, serpentine, or the like and can optionally include branches into multiple channels or can connect with other channels.

In an aspect, the porous silicon can be thermally carbonized and/or oxidized and then the binding agent can be attached to the carbonized surface or oxidized surface of the porous silicon. The binding agent can be attached to the carbonized surface or oxidized surface of the porous silicon using appropriate chemistry, for example, the bonding that is described herein. As a result, the binding agent is indirectly attached to the porous silicon membrane.

In an aspect, the porous membrane can have a length and width or diameter of a few millimeters to a few centimeters. The porous membrane can have a thickness of about 0.5 to 50 μm, about 10 to 50 μm, about 10 to 50 μm, about 10 to 30 μm, or about 20 μm.

In an aspect, the porous membrane includes a plurality of pores. The pores extend through the porous membrane so that the sample fluid can flow through the porous membrane and into the absorbent layer. Each of the pores of the plurality of pores independently can have a diameter of about 1 to 100 nm, about 20 to 80 nm, about 30 to 70 nm, about 50 to 100 nm, or about 50 to 80 nm. While each of the pores of the plurality of pores may not have the substantially same diameter but have about the same diameter (e.g., the diameter of the pores is within about 10%, about 20%, or about 30% of the median diameter of the plurality of the pores). In an aspect, the cach of the pores of the plurality of pores is substantially (e.g., less than 5%deviation from the median diameter of the plurality of the pores) the same.

The porous membrane can be stratified or unstratified. When the porous membrane is unstratified, the diameter of the pores is about the same or are substantially the same through the thickness of the porous membrane. When the porous membrane is stratified, the stratified porous membrane has at least two strata (e.g., 2 to 40 strata, 2 to 10 strata, 3 to 12 strata, 3 strata, 5 strata, 7 strata, 9 strata, or 11 strata) and cach strata have a plurality of pores having a diameter that is different by at least 5 nm (e.g., about 5 to 30 nm, about 5 to 20 nm, about 5 to 10 nm, or about 5 to 7 nm) or at least 10 nm (e.g., about 10 to 30 nm, about 10 to 20 nm, or about 10 to 15 nm). In an aspect, the number of strata is odd, where the middle stratum has the narrowest diameter and strata on either side of the middle stratum have larger diameter pores such that the strata furthest away from the middle stratum have the pores with the largest diameter.

For example, the stratified porous membrane can have a first stratum, a second stratum, and a third stratum. The second stratum is the middle stratum. In other words, the second stratum is positioned between the first stratum and the third stratum. The first stratum has a first plurality of pores, the second stratum has a second plurality of pores, and the third stratum has a third plurality of pores. The first plurality of pores and the third plurality of pores have about the same diameter, where the second plurality of pores have a smaller diameter than the first plurality of pore and the second plurality of pores. In an aspect, the first stratum and the third stratum can have a thickness of about 0.5 to 3.5 μm or about 0.5 to 2 μm and the second stratum has a thickness of about 0.5 to 50 μm. In an aspect, the first plurality of pores has a diameter of about 50 to 80 nm and the third plurality of pores has a diameter of about 50 to 80 nm. The diameter of the pores in the first plurality of pores and the third plurality of pores are the same. The second plurality of pores has a diameter of about 20 to 40 nm.

In an aspect, the porous membrane is a porous silicon membrane. While the following is directed to porous silicon, the same or similar process can be directed to other materials that can be used to make the porous membrane. The porous silicon membrane can be fabricated by electrochemical etch of p-type or n-type Si wafer in hydrofluoric acid. Changing the applied current density during the electrochemical etch can change the porosity and pore size, and can therefore stratify a porous silicon film with two or more strata. The membrane can be “lifted-off” the silicon substrate by applying high-current density pulses to detach it. In order to decrease the water contact angle and prepare the surface for further chemical modification steps, PSi free-standing membranes were thermally oxidized at 500° C. Next, the PSi membrane was transferred to a nitrocellulose (NC) membrane (or other membrane support), for example, via wet transfer in water. The PSi membrane was added to the Petri dish, and then the water was slowly removed with a pipettor until the PSi membrane touched and formed a good contact with the NC membrane. The PSi-NC system was allowed to dry in air. The good adhesion between the two membranes is primarily attributed to electrostatic interactions. Wax-printed paper (top cover) patterned with a small circular opening, to allow optical measurements, was attached as a top layer to the PSi/NC membrane system using double-sided tape. An absorbent layer was then attached to the bottom of the wax-printed NC membrane using double-sided tape. Finally, to ensure that analyte does not leak through the bottom of the absorbent layer, fully-filled wax-printed paper was attached to the backside of the absorbent layer as the final layer of the PSi-on-paper system.

In an embodiment, the porous membrane can include surface moicties that can bond with the binding agent. In an embodiment, the porous membrane can include organically modified moieties (e.g., hydroxyl groups, carboxylate groups, amines, phosphoric acid, sulfonic acid, thiols, phosphines, zwitterions, and the like) on the surface (e.g., outside and/or inside surfaces of pores) of the porous membrane. In an embodiment, the porous membrane can include surface hydroxyl groups, carboxylate groups, amines, phosphoric acid groups, sulfonic acid groups, thiols, phosphines, zwitterions, and the like, that the binding agent can directly covalently bond and/or indirectly covalently bond (e.g., covalently bond to a linker covalently bonded to the porous membrane).

In an aspect, the binding agent has an affinity for the target, where the target can be present in a sample fluid such as saliva, blood, and urine. In another example, the fluid sample can be from an environmental source such as sewage (e.g., detect viral levels), lake or river (e.g., detect contaminants such as pesticides, herbicides, heavy metals, and the like), or ocean (e.g., detect red tides). The binding agent can include a chemical agent or a biological agent. In an aspect, the biological agent can include: a protein, an antibody (monoclonal or polyclonal), an antigen, a polynucleotide, an enzyme, a hapten, a polysaccharide, a sugar, a fatty acid, a steroid, a glycoprotein, a carbohydrate, a lipid, a purine, a pyrimidine, an aptamer, a small molecule, a ligand, or combinations thereof, where the binding agent has an affinity for a target.

The term “affinity” can include biological interactions and/or chemical interactions. The biological interactions can include, but are not limited to, bonding or hybridization among one or more biological functional groups located on or within (e.g., lysing a cell) the biological target and/or the capture agent. The chemical interaction can include, but is not limited to, bonding among one or more functional groups (e.g., organic and/or inorganic functional groups) located on the binding agent. The biological interactions and/or chemical interactions can be direct or indirect (e.g., using a linking group such as that described herein or other appropriate linking group). In an aspect, the binding agent has a strong preference (e.g., 90% or more, 95% or more, 99% or more, or 99.9% or more) to bond with the target over other components that might be present in the sample fluid so that the binding agent is an effective way to sense and detect the presence of the target in the samples of interest. The binding agent can be associated with target through the biological interactions and/or chemical interactions. The target can be bound to the binding agent, where the term “bound” can include biological interactions and/or chemical interactions described above and include the meaning of “bound” as provided below, where the interaction or bond depends upon the target and the binding agent.

In general, the target can be viruses, algae, parasites, archaea, protozoa, fungi, spores, apicomplexan, trematodes, nematodes, mycoplasma, small molecules (e.g., heavy metals, pyrocatechol, glucose, ochratoxin A, aflatoxin B1, Fumonisin B1, and the like), oligonucleotides (e.g., DNA, miRNA), peptides (e.g., glutathione, insulin, and the like), proteins (e.g., trypsin, ATP, protein A, streptavidin, hydatid disease biomarker, cystic hydatid discase antigen, BSA, TNFalpha, C-reactive protein, thrombin, prostate specific antigen, human kallikrein 2, and the like), enzymes (e.g., sortase A and MMPs, and the like), bacteria (e.g., E. coli, L. acidophilus and the like), lipids, carbohydrates, metabolites, hormones, antigens, antibodies, and glycoproteins. In an aspect, the target can be viruses, archaea, mycoplasma, small molecules (e.g., heavy metals, pyrocatechol, glucose, ochratoxin A, aflatoxin B1, Fumonisin B1, and the like), oligonucleotides (e.g., DNA, miRNA), peptides (e.g., glutathione, insulin, and the like), proteins (e.g., trypsin, ATP, protein A, streptavidin, hydatid disease biomarker, cystic hydatid disease antigen, BSA. TNFalpha, C-reactive protein, thrombin, prostate specific antigen, human kallikrein 2, and the like), enzymes (e.g., sortase A and MMPs, and the likc), bacteria (e.g., E. coli, L. acidophilus and the like), lipids, carbohydrates, metabolites, hormones, antigens, antibodies, and glycoproteins. In an aspect, the target is a virus such as influenza viruses, coronaviruses, rhinoviruses, and adenoviruses. In an aspect, a microorganism can be treated (e.g. lysed) so that the a specific target (e.g., protein, DNA, RNA, receptor binding domain (e.g. spike protein), and the like) to the microorganism can be in the fluid sample. For example, a microorganism that has dimensions that do not permit the microorganism to enter the pores can be treated so that a specific target can be present in the fluid sample and detected. In this way, the target (e.g., associated with a microorganism or a larger chemical compound) can be indirectly detected.

The binding agent can be bonded to the surface of the pores of the porous membrane. The term “bound”, “bond”, or “bonded” can include, but is not limited to, chemically bonded (e.g., covalently or ionically), biologically bonded, biochemically bonded, and/or otherwise associated with the particle. In an embodiment, “bound”, “bond”, or “bonded” can include, but is not limited to, a covalent bond, a non-covalent bond, an ionic bond, a chelated bond, as well as being bound through interactions such as, but not limited to, hydrophobic interactions, hydrophilic interactions, charge-charge interactions, π-stacking interactions, combinations thereof, and like interactions. The binding agent can be bonded directly or indirectly (e.g. a linking group (e.g., silicon linker such as a silanol group (e.g., a siloxane linkage))) to the surfaced of the pores of the porous membrane.

In an aspect, a linking group is a bi-functional molecule that can bind to the surface of the porous membrane at one point and to the binding agent at another point. In an aspect, the linking group can be an organosilane with a reactive atom on a carbon chain (e.g., —(CH₂)ₙ—) such as, but not limited to, N, S, P, or O or include reactive groups such as alkenes or alkynes. In another aspect, the linking group can be a bi-functional hydrocarbon backbone chain (e.g., —(CH₂)ₙ—), where the hydrocarbon backbone chain can be saturated or unsaturated and could include branching.

The present disclosure also provides for a method detecting the presence of a target in the fluid sample. The method can be used to detect the presence of a target or the method can be used to quantify the amount of target present in the fluid sample. In an aspect, the method includes exposing a sample fluid on the porous membrane of the sensor as described herein. In an embodiment, the sample fluid is disposed directly on the porous membrane, while in another embodiment the sample fluid can be flowed via lateral flow to the porous membrane. For example, the sample fluid can be disposed directly on the porous membrane through the opening of the top cover. In another example, the sample fluid is disposed onto a channel through the opening of the top cover and the fluid sample lateral flows to the porous membrane. The porous membrane includes binding agents on the surface of the pores of the porous membrane that have an affinity for at least one target. As the fluid sample flows through the pores of the porous membrane, the components of the fluid sample are exposed to the binding agent. If the sample fluid includes the target as one of its components, the target will be associated with (e.g., biological interactions and/or chemical interactions such as bonding as described herein) the binding agent and produce a color change in the porous membrane. If the sample fluid does not include the target, the color change does not occur. The porous membrane undergoes the color change that can be detected visually, using a spectrometer, or an image capture device. In an aspect, the image capture device is a camera phone or other image capture device and the image can then be processed using an application on the phone or remotely to determine if the color change is such that it is a positive indication of the target being present in the sample fluid. In an aspect, the color change can be compared to a standard or calibration curve. In an aspect, the color change can be correlated to a certain amount of the target present in the fluid sample. For example, the degree of the color change can be compared to a calibration curve (e.g., in an application or other program) to determine the amount of target present in the fluid sample.

In another embodiment, the porous membrane can include multiple types of binding agents that are specific to different targets. For example, one type of binding agent can have affinity for a corona virus and another type of binding agent can have an affinity for a flu virus. When the fluid sample flow across the two different types of binding agents, if one or both targets are in the fluid sample, one (e.g., flu virus), two (e.g., corona virus), or three (e.g., both the corona virus and the flu virus) different types of color changes can occur. The color change can be detected visually (e.g., one of three colors), using a spectrometer, or an image capture device. In an aspect, the image capture device is a camera phone or other image capture device and the image can then be processed using an application on the phone or remotely to determine if the color change is such that it is a positive indication of the flu virus, corona virus, or both the flu virus and the corona virus being present in the sample fluid. In an aspect, the color change can be compared to a standard or calibration curve to determine the target present in the fluid sample.

### EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### Example 1

Development of a reliable rapid diagnostic test (RDT) for viral infections that is low-cost, widely deployable, highly sensitive, and provides results in less than 30 minutes is crucial for limiting the spread of infection. The most common form of RDT is a paper-based immunoassay. However, these paper-based tests face challenges in achieving sufficient sensitivity to detect diseases at an early state and providing quantification of the detected biomarkers.

Porous silicon (PSi) is an advantageous material for quantitative optical biosensing with high sensitivity detection of a variety of biomolecules but, to date, has not been translated for use in RDTs. Here, we report the incorporation of PSi on paper toward the realization of a PSi-based RDT with the capability for quantification of detected molecules. Following traditional electrochemical etching methods to form PSi thin films, the PSi was detached from the silicon substrate by applying a series of high current density pulses. Appropriate methods must be followed to minimize potential cracking and bending of the PSi and to ensure robust adhesion of the PSi membrane on paper. Solutions of different concentrations of bovine serum albumin were exposed to the PSi-on-paper platform and real-time optical reflectance measurements were taken to benchmark the sensor. COMSOL finite element method simulations were carried out to investigate the mass transport and adsorption kinetics in the PSi-on-paper platform. Both simulations and experiments show a response-time dependence on PSi pore size.

In this example, we explore a porous silicon (PSi)-on-paper optical biosensor with the potential to achieve rapid, accurate, quantitative, and high sensitivity detection of biomarkers that may significantly advance the capabilities of RDTs. PSi has already been demonstrated as a cost-effective, case-to-use platform for optical biosensing of proteins, DNA, and other small molecules [2]. Recent work to reduce the detection limit of PSi optical biosensors [3,4], allow operation in complex media [5,6], and realize quantitative readout on a smartphone [7] suggests that PSi optical biosensors are well-positioned to have a positive impact on rapid diagnostic technology if further advances are made to enable the integration of PSi in a portable, small form factor diagnostic.

### Method and Results

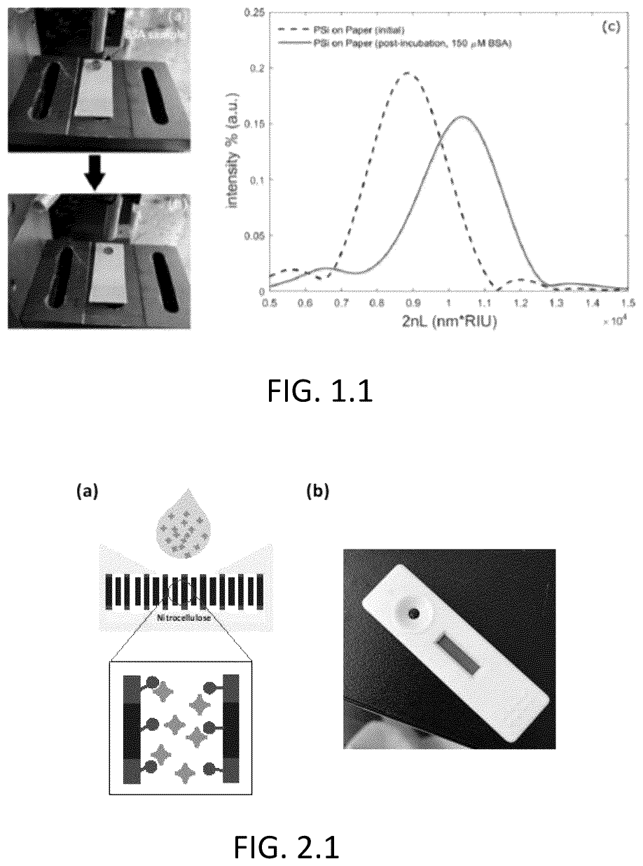

PSi thin films were formed by electrochemical etching of highly doped p-type silicon wafers in a solution of 15% hydrofluoric acid in ethanol. The PSi films were subsequently removed from the silicon substrate by electropolishing with a series of high current density pulses. The thickness of the porous silicon film and membrane lift-off conditions must be chosen carefully to ensure that the PSi membrane does not crack during the lift-off process. We note that careful handling is required during all processing, including the transfer of the PSi membrane to the paper substrate. FIG. 1.1(a-b) shows the assembled PSi-on-paper platform. Solutions of different concentrations of bovine serum albumin (BSA) were exposed to the PSi-on-paper platform and real-time optical reflectance measurements were taken to characterize the response of the sensor. The reflectance spectra of the PSi membranes are characterized by the expected Fabry-Perot fringes. The characteristic frequency of the fringes is determined by the optical thickness of the film, which changes when BSA is adsorbed in the pores. Taking a fast Fourier transform (FFT) of the reflectance spectrum reveals the optical thickness of the film [8]. FIG. 1.1b shows how the optical thickness of the PSi membrane changes when a 150 μM BSA solution is exposed to the PSi-on-paper sensor. The magnitude of this signal change can be directly correlated to the BSA concentration. Both experiments and COMSOL finite element method simulations show that the magnitude and response time of the PSi-on-paper sensor depend on PSi pore size and other parameters of the PSi-on-paper platform.

### Conclusions

We report the incorporation of PSi into a paper-based sensing system and demonstrate feasibility through real-time optical reflectance measurements that monitor BSA adsorption in the pores. Careful fabrication protocols are necessary to ensure robust and repeatable assembly of the PSi-on-paper platform. Experiments and COMSOL finite element method simulations show how the performance of the PSi-on-paper sensor depends on the physical and chemical parameters of the materials comprising the sensor. This platform has the potential to significantly improve the capabilities of RDTs.

### REFERENCES FOR EXAMPLE 1

[1] A. M. Caliendo, D. N. Gilbert, C. C. Ginocchio, K. E. Hanson, L. May, T. C. Quinn, F. C. Tenover, D. Alland, A. J. Blaschke, R. A. Bonomo, K. C. Carroll, M. J. Ferraro, L. R. Hirschhorn, W. P. Joseph, T. Karchmer, A. T. MacIntyre, L. B. Reller, and A. F. Jackson, “Better tests, better care: Improved diagnostics for infectious diseases,” Clin. Infect. Dis. 57, S139 (2013).
[2] S. Arshavsky-Graham, N Massad-Ivanir, E. Segal, and S. M. Weiss, “Porous silicon-based photonic biosensors: Current status and emerging applications,” Anal. Chem. 91, 441 (2019).
[3] S. Mariani, L. Pino, L. M. Strambini, L. Tedeschi, and G. Barillaro, “10 000-fold improvement in protein detection using nanostructured porous silicon interferometric aptasensors,” ACS Sens. 1, 1471 (2016).
[4] S. J. Ward, R. Layouni, S. Arshavsky-Graham, E. Segal, and S. M. Weiss, “Morlet wavelet filtering and phase analysis to reduce the limit of detection for thin film optical biosensors,” ACS Sens. 6, 2967 (2021).
[5] S. Arshavsky-Graham, N. Massad-Ivanir, F. Paratore, T. Scheper, M. Bercovici, and E. Segal, “On chip protein pre-concentration for enhancing the sensitivity of porous silicon biosensors,” ACS Sens. 2, 1767 (2017).
[6] R. Chhasatia, M. J. Sweetman, F. J. Harding, M. Waibel, T. Kay, H. Thomas, T. Loudovaris, and N. H. Voelcker, “Non-invasive, in vitro analysis of islet insulin production enabled by an optical porous silicon biosensor,” Biosens. Bioelectron. 91, 515 (2017).
[7] T. Cao, Y. Zhao, C. A. Nattoo, R. Layouni, and S. M. Weiss, “A smartphone biosensor based on analysing structural colour of porous silicon,” Analyst 144, 3942 (2019).
[8] C. Pacholski, M. Sartor, M. J. Sailor, F. Cunin, and G. M. Miskelly, “Biosensing using porous silicon double-layer interferometers: Reflective interferometric Fourier transform spectroscopy,” J. Am. Chem. Soc. 127, 11636 (2005).

### Example 2

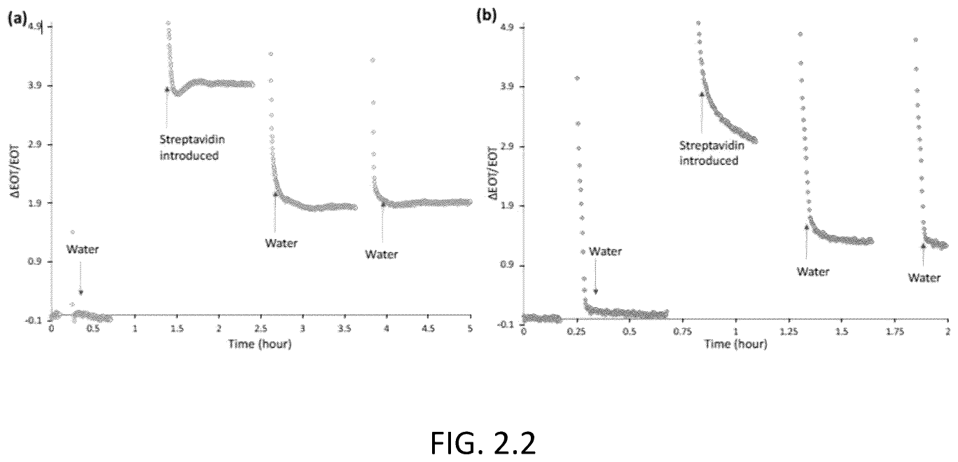

Rapid diagnostic tests (RDTs) are indispensable in healthcare, providing quick and accessible results for various medical conditions. While paper-based immunoassays are prevalent in RDTs due to their affordability and user-friendly nature, challenges persist, particularly in terms of sensitivity variations and difficulties in quantifying color changes [1].These limitations drive the ongoing evolution of RDT platforms towards more reliable, sensitive, and quantitative detection methods. Here we suggest the integration of PSi membranes with paper-based substrates as a potential path forward to achieving quantitative RDTs with low detection limits (FIG. 2.1). PSi is a promising material for biosensing due to its high surface area for molecular capture, straightforward optical readout, cost-effectiveness, and biocompatibility. Benchtop PSi biosensors have been demonstrated for the detection of a variety of species including proteins, DNA, and other small molecules in both buffered and complex media [2,3]. Interestingly, for incorporation into a paper-based RDT, a kcy challenge facing traditional PSi biosensors, namely mass transport in closed-ended pores on a silicon substrate [4], can be mitigated. It has been shown that using an open-ended PSi membrane [5] enables improved mass transport and faster response time. As demonstrated by numerous paper-based lateral flow assays, a paper-based substrate can drive analyte flow without the need for traditional microfluidic cells and an external pump [6]. Hence, the incorporation of a PSi FSM on a paper-based substrate offers the opportunity to combine the biosensing advantages of the PSi material system with those of paper-based assays. Importantly, PSi biosensors have been shown to be compatible with smartphone readout [7], opening the door to a broadly accessible method for quantitative readout of PSi paper-based biosensors.

### Methods

PSi FSMs were fabricated by electrochemical etching of crystalline silicon in a hydrofluoric acid-based electrolyte, followed by an electropolishing “liftoff” step to remove the PSi film from the substrate. In particular, a sacrificial layer of PSi is first etched at a current density of 80 mA/cm² for 100 s in an electrolyte solution of a 3:7 volume ratio of hydrofluoric acid (HF) and EtOH (100%) and is subsequently removed with a NaOH solution (1:9, 1M NaOH to EtOH). After the sacrificial layer had enough time to dissolve, the Si surface was washed with DI water and EtOH to remove residual NaOH. Then, a specific current density profile was applied to create the desired PSi structures, featuring varying pore sizes and thicknesses as a function of depth in the film: current densities of 80 mA/cm² for 42.32 s, then 40 mA/cm² for 846.4s, followed by 80 mA/cm² for 42.32 s were applied. A PSi free-standing membrane was obtained using a lift-off process in which a high current density of 500 mA/cm² for 1.7 s was applied two times. The PSi membrane was then thermally oxidized at 500° C. with care taken to mitigate thermal shock by implementing relatively long ramp up and ramp down times. Subsequently, the thermally oxidized PSi membrane was immersed in a 4% solution of (3-aminopropyl)triethoxysilane (APTES) (99%) for 10 minutes within a glass petri dish. It was then soaked in methanol for 15 minutes to eliminate any unreacted APTES. After thorough drying, the PSi membrane underwent annealing in an oven at 150° C. for 15 min. Following the annealing step, the PSi membrane was submerged in a solution of 0.01 mg/mL sulfo-NHS-biotin in phosphate-buffered saline (PBS) and incubated overnight. Finally, the biotin-conjugated PSi membrane was subjected to a 1-hour soak in water and ethanol to remove any residual biotin and subsequently dried on an absorbent pad.

After functionalization, the PSi membrane was ready to be assembled into a test cartridge. The biotin-conjugated PSi membrane was first affixed onto a nitrocellulose (NC) substrate in initial tests but the NC substrate was not included in subsequent tests. Wax-coated paper with an aperture was placed atop the PSi FSM to serve as a reference point for the sensor measurement. To facilitate the withdrawal of solutions from the PSi membrane, one or more absorbent pads were positioned beneath the NC layer in the initial tests and beneath the PSi membrane in subsequent tests. A plastic cartridge was employed to hold everything together and minimize any air gaps between different materials (FIG. 2.1b).

### Experimental Results and Discussions

Streptavidin sensing was conducted in real-time by exposing the biotin-functionalized PSi on paper test to a 10 μL solution of streptavidin with continuous recording of the reflectance spectrum. FIG. 2.2 shows the real-time effective optical thickness (EOT) changes of the PSi sensor during the streptavidin sensing experiment; the reflective interferometric Fourier transform spectroscopy method was used to convert the measured reflectance spectra to EOT data [8]. The baseline was acquired following an initial wetting of the PSi sensor with water. A notable increase in EOT was observed upon introduction of the streptavidin solution, in part due to streptavidin-biotin binding, followed by a subsequent decrease in EOT after the first water wash step that removed unbound species. No additional change in EOT occurred after a second wash step. The overall change in EOT from the baseline measurement to the final measurement is directly correlated with the number of streptavidin molecules attached in the pores. Notably, in the first experiment shown in FIG. 2.2a, it takes about 30 min for the PSi to dry and for the optical signal to reach equilibrium after each exposure to water or protein. To investigate what minimum response times are possible in an optimized PSi on paper test, a fan was introduced as a means of accelerating the solvent evaporation process. When the streptavidin sensing experiment was repeated with the implementation of the fan, the response time was reduced to approximately 15 min. We anticipate that changes to the absorbent pad parameters and potentially to the PSi membrane design as well will expedite the drying process without the use of a fan.

### Conclusions

We demonstrated the potential of PSi as a quantitative, paper-based RDT platform for the real-time sensing of specific analytes. PSi membranes were functionalized with biotin, affixed to paper-based substrates, and assembled in a cartridge for the detection of streptavidin molecules. A clear change in EOT was measured, confirming streptavidin attachment. Ongoing studies on the flow dynamics in the PSi on paper sensor are expected to lead to design improvements that will enable rapid response times (<20 min) without the need for an external method of drying the sensor. The combination of the unique optical properties of PSi and its compatibility with paper-based platforms and smartphone readout, holds promise for the development of cost-effective, widely accessible, and highly quantitative biosensors for a wide range of applications.

### REFERENCES FOR EXAMPLE 2

[1] R. Luo, N. Fongwen, C. K-Cirino, E. Harris, A. W-Smith, RW. Peeling, Clin. Micr. Infe. 25, 659 (2019).

[2] S. A-Graham, N M-Ivanir, E. Segal, S. M. Weiss, Anal. Chem 91, 441 (2019)
[3] R. Moretta, L. De Stefano, M. Terracciano, I. Rea, Sensors 21, 1336 (2021).
[4] S. A-Graham, E. Boyko, R. Salama, E. Segal, ACS Sens. 5, 3058 (2020).
[5] Y. Zhao, G. Gaur, S. T. Retterer, P. E. Laibinis, S. M. Weiss, Anal. Chem. 88, 10940 (2016).
[6] E. Noviana, T. Ozer, C. S. Carrell, J. S. Link, C. McMahon, I. Jang, C. S. Henry, Chem. Rev. 121, 11835 (2021).
[7] T. Cao, Y. Zhao, C. A. Nattoo, R. Layouni, S. M. Weiss, Analyst 144, 3942 (2019).
[8] C. Pacholski, C. Yu, G. M. Miskelly, D. Godin, M. J. Sailor. J. Am. Chem. Soc. 128, 4250 (2006).

### Example 3

Development of a reliable rapid diagnostic test (RDT) for viral infections that is low-cost, widely deployable, highly sensitive, and provides results in less than 30 minutes is crucial for limiting the spread of infection and creating a large database of information about disease progression and spread for epidemiological studies.¹ With the emergence of COVID-19, it has become readily apparent that further research is needed to meet demands for testing during the current pandemic as well as for future disease outbreaks.²⁻⁴ In general, RDTs face challenges in achieving sufficient sensitivity to detect diseases at an early stage and most RDTs lack the capability to quantify the number of biomarkers present in a test sample.⁵ Porous silicon has the potential to be adapted to rapid diagnostic tests, but improvements in surface stability and resistance to fouling are crucial before such transition. Additionally, mass transport challenges limit the development of porous silicon (PSi) for real disease diagnostics. Thus, novel configuration needs to be implemented in order to overcome diffusion limitations and enhance the overall sensitivity of PSi biosensors.

### Rapid Diagnostic Tests (RDT)

RDTs are widely used to detect a variety of diseases and other conditions (e.g., influenza, malaria, or pregnancy), and have proven to be effective and widely accessible in clinics, hospitals, and even homes, in some cases.⁵⁻⁸ For disease detection, RDTs enable carly pathogen identification and treatment, which facilitates timely disease containment efforts. The specificity of most commercialized RDTs is high and is similar among commercially available products; however, the sensitivity of RDTs varies over a wide range.^{1,9-12} Moreover, it is usually not possible to diagnose asymptomatic disease carriers using RDTs because the detection limits are not sufficiently low.^{1,13} The most common form of RDT is paper-based immunoassays. These are paper-based lateral flow assays (LFA) that are cheap and easy to use. Commonly used LFAs include pregnancy tests, which detect the levels of human chorionic gonadotropin (hCG) hormone in urine and have an LOD of 25-200 IU/mL.14 Another example are antigen tests for infectious disease detection such as the flu. For instance, Influenza A virus can be detected by capturing its nucleoprotein (NP) in a lateral flow test, with a limit if detection as low as 250 ng/mL.¹⁵ LFAs can also be used in the detection of foodborne pathogens such as E.coli. (LOD ˜3000-6000 cells).¹⁶ In these devices, a liquid travels across a nitrocellulose membrane by capillary action, carrying a patient's biological sample (e.g., blood or urine) mixed with an enzyme/dye tagged mobile detection agent (e.g., antibody) that binds to a desired target analyte in the biological sample (e.g., antigen). The target/detection agent complex attaches to specific capture agents immobilized on a dedicated test line in the detection zone of the LFA, causing a color change to occur. A nearby control line is also often utilized to confirm there is proper liquid flow through the paper strip. Quantification of the color change is challenging, if not impossible, in many LFAs. I note that while many RDTs for viral infections rely on immunoassay-based detection, RDT platforms are also being developed to realize faster and more cost-effective molecular-based detection such as nucleic acid amplification.^{6,17} Because RDTs struggle to identify asymptomatic disease carriers and quantify analytes that are disease indicators, there remains an untapped opportunity to enhance the capabilities of RDTs by combining their positive attributes (low-cost, simple, rapid, portable) with higher sensitivity and quantification offered by label-free benchtop sensors such as PSi sensors.

### Porous Silicon Optical Biosensing

PSi has been demonstrated as a promising material for benchtop label-free optical biosensing applications (e.g., DNA, ^{18,19-21} proteins, ²²⁻²³ toxins,²⁵ and a variety of other small molecules²⁶⁻²⁸) for more than two decades due in large part to the simplicity of measuring changes in the optical properties of PSi that directly correlate to the quantity of molecules captured in the material. Another important advantage of PSi for biosensing applications is its high internal surface area within a small areal footprint (e.g., >100 m²/cm² is achievable),²⁹ which enables more target molecules to be readily captured from a given volume of solution, leading to improved detection sensitivity. Even when only considering a small sensor footprint of 1 mm×1 mm, the available surface area for probe molecule immobilization and target molecule capture is 100-fold larger for PSi sensors compared to flat surface sensors. In addition, PSi is cost-effective, easy to fabricate in a scalable manner, and compatible with a wide range of functionalization chemistries.³⁰ While optical measurements can be carried out in a straightforward manner with a white light source and spectrometer on a benchtop, to date, there has been no path forward demonstrated for transitioning the fundamental principles of label-free optical biosensing using PSi to a platform that meets criteria for RDTs and point-of-care diagnostics. Of particular interest, is the use of PSi for the detection of antigens and antibodies in both buffer solutions and complex media. ³¹⁻³⁴ With proper surface preparation including antifouling coatings, PSi sensors can operate in a complex medium, such human serum and potentially other biological media. This brings this platform closer to implementation in real-life clinically relevant rapid diagnostics. Furthermore, one recent advance in this direction is Cao et al.'s demonstration of replacing the white light source and spectrometer with a smartphone LED and camera³⁵. In summary, while label-free PSi optical biosensors have great potential for straightforward, highly sensitive, quantitative diagnostic measurements, translating PSi sensors to a cost-effective, widely deployable RDT platform for point-of-care testing remains an unexplored opportunity.

### Mass Transport in Porous Silicn

One of the key advantages of PSi biosensors—their large active sensing surface area—can also be a key bottleneck if the pore diameters are not sufficiently large compared to the size of the probe and target molecules. Mass transport is thus a critical consideration in the design of PSi biosensors.

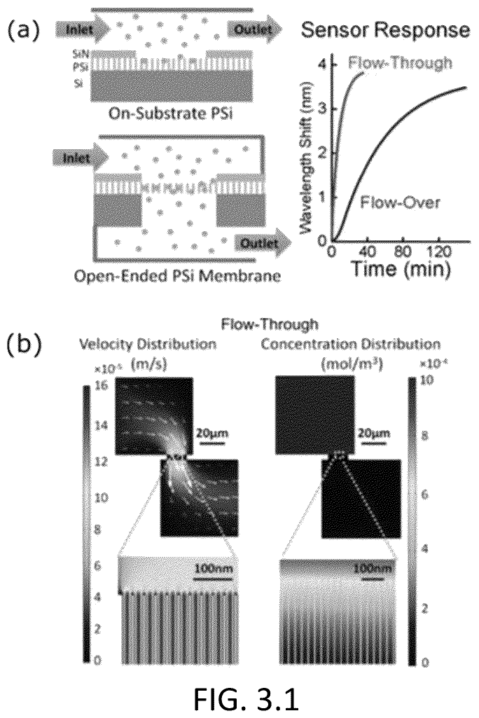

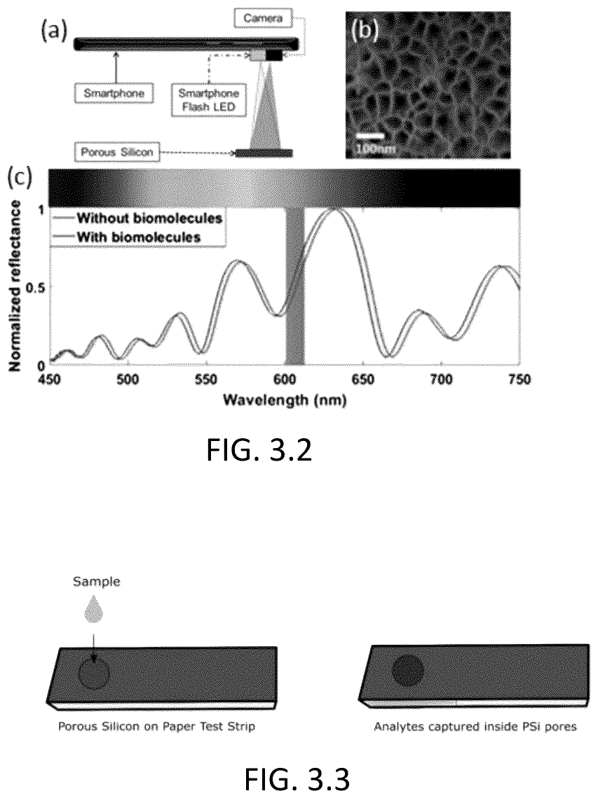

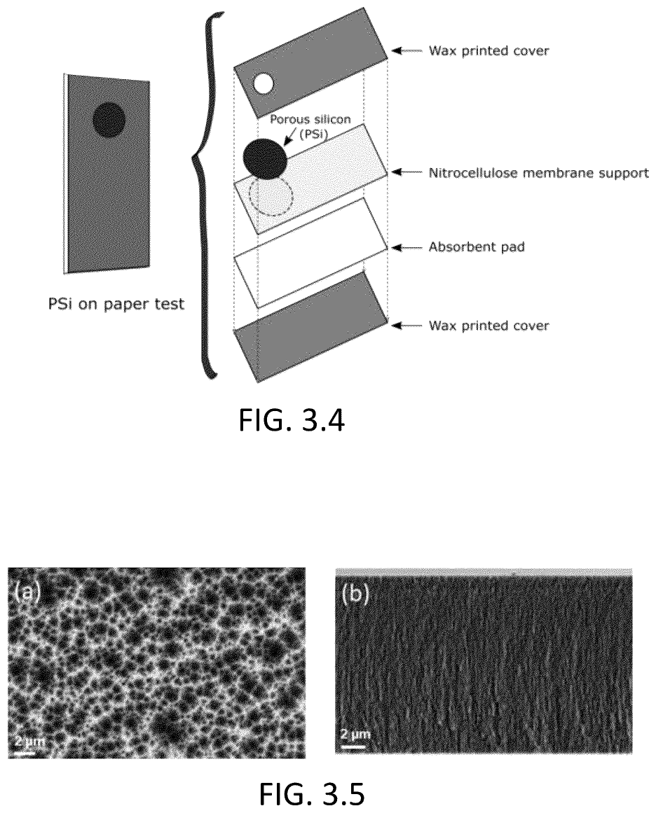

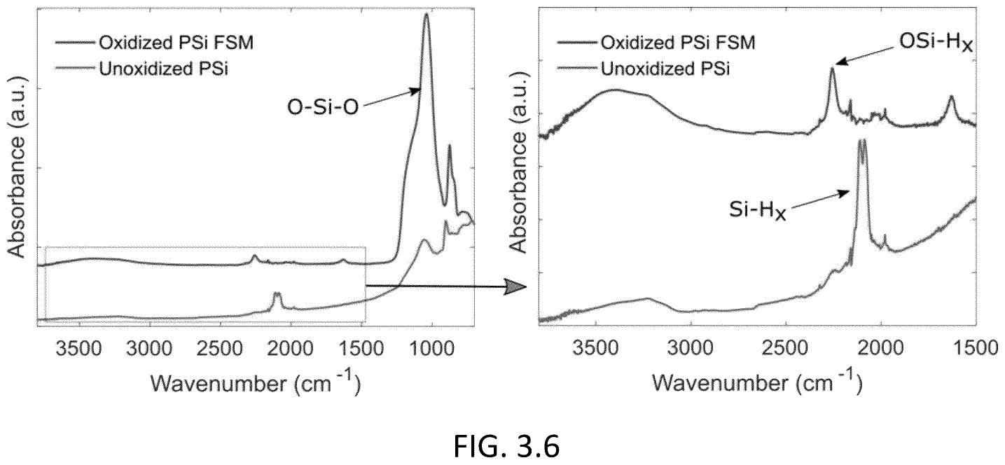

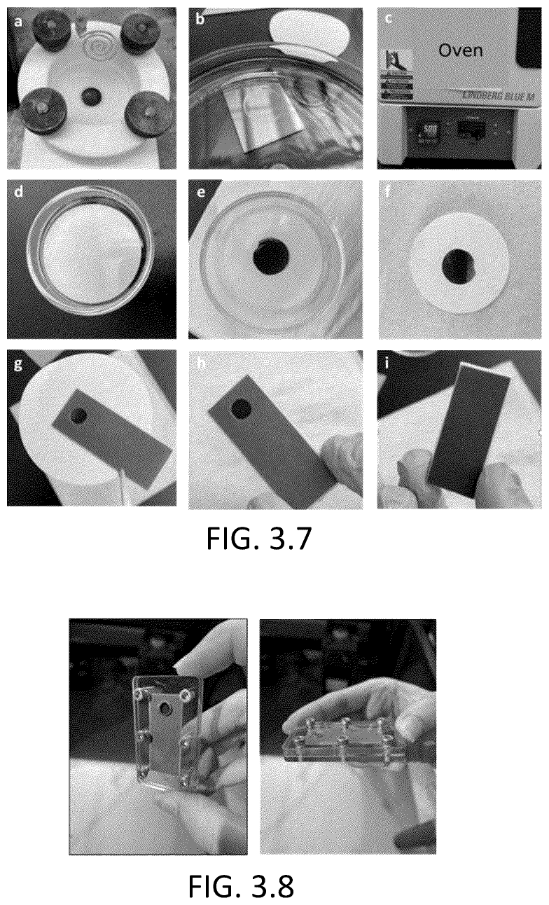

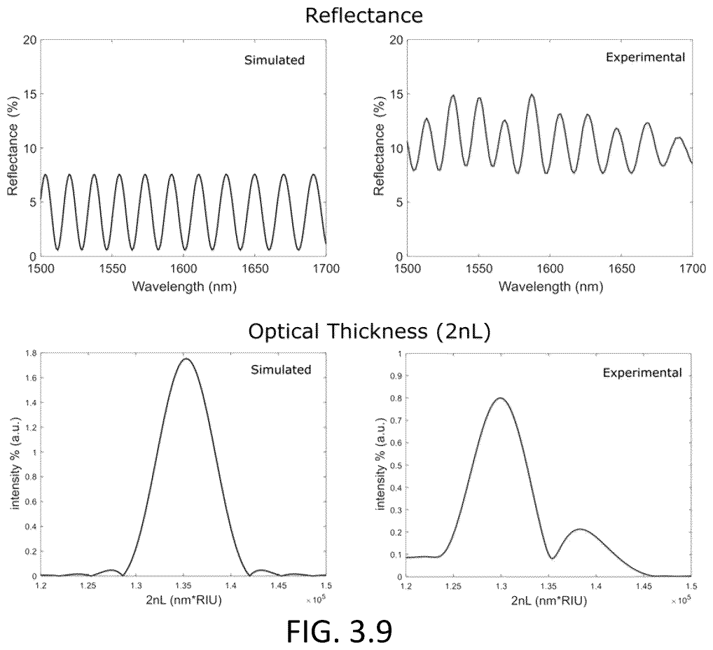

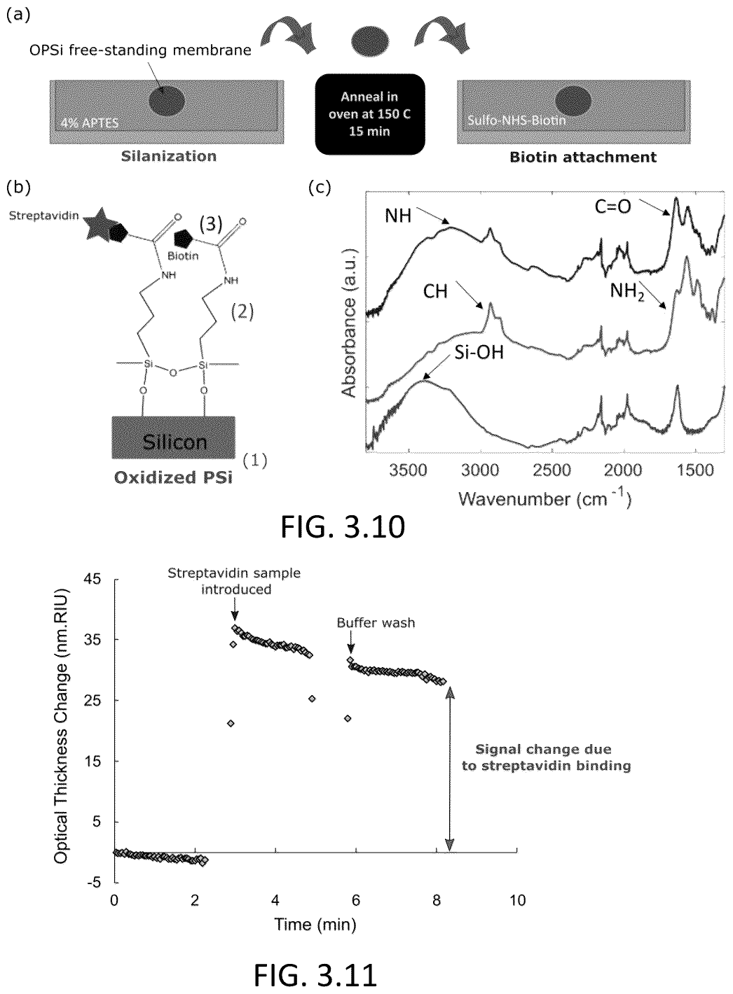

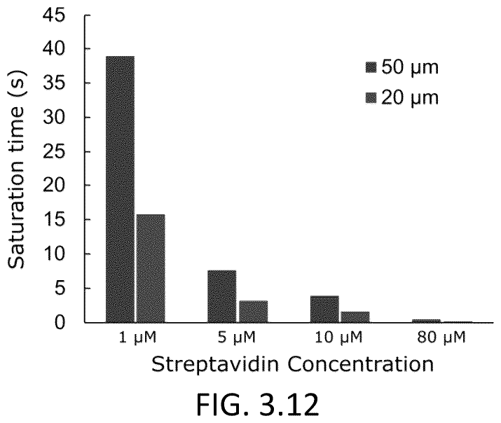

Zhang et al. have carried out careful simulations and measurements to understand mass transport in closed-ended flow-over and open-ended flow-through PSi sensor schemes, as illustrated in FIG. 3.1.³⁶ Using the finite element method software COMSOL Multiphysics under the assumption of steady-state 2D laminar flow, Zhang solved the Navier-Stokes and convection-diffusion equations to calculate the velocity profile and concentration distribution of analyte solution in PSi films and membranes. The adsorption kinetics of target species of interest were then combined with mass transport in the PSi region to determine the expected response time of the system. These results showed a 6-fold enhancement in response time for the detection of streptavidin molecules, along with faster transport rates, when using the flow-through PSi membrane in comparison to the flow-over PSi film. This enhancement in sensitivity combined with fouling and corrosion resistance surface can lead to the desired realization of a highly robust and sensitive PSi rapid test for infectious disease, such as malaria.

### Smartphone Detection with Porous Silicon Optical Biosensors

Most label-free PSi optical biosensors utilize a benchtop spectrometer for detection of the optical signal. Therefore, advances in the measurement configuration are needed to transition PSi optical sensor systems to a design that is compatible with RDTs. As a first step, Cao et al. recently demonstrated the use of a smartphone for optical readout of a label-free PSi microcavity sensor.³⁵

The LED flash and camera of the smartphone replace the benchtop white light source and detector, and an intensity-based measurement replaces the spectral measurement shown in FIG. 3.2b. As illustrated in FIG. 3.2, the shift in the reflectance spectrum that results from molecular attachment in PSi can be correlated to an intensity change measured within a narrow spectral bandwidth corresponding to the red pixels in the camera image sensor. For a large enough refractive index change of the PSi film, for example due to infiltration of the pores with ethanol, the resulting color change of the PSi microcavity can be seen by eye. However, for small refractive index changes of the PSi film, for example from the capture of a low concentration of target molecules, only a slight color change of the PSi microcavity results and quantification requires analysis of camera images. Accordingly, the integrated intensity within the measurement bandwidth is compared before and after molecular attachment; quantification is achieved through comparison to a calibration curve. The feasibility of the PSi-smartphone sensor was shown with a biotin-streptavidin assay.³⁷ The limit of detection was estimated to be 500 nM with a resolution similar to that of an Ocean Optics spectrometer. While the reported approach allows the direct use of smartphones with negligible modification for measurements, smartphone spectrometers that require a small accessory also could be utilized for measurement of PSi sensors.^{38,39} Past work with smartphone measurements of PSi sensors establishes the feasibility of a RDT-compatible, quantitative readout for the PSi-on-paper sensor investigated here.

### Porous Silicon on Paper RDT

Here, we build on prior studies of PSi biosensors, including methods for robust surface passivation and smartphone optical readout, ^{35,20,21} and numerical modeling of mass transport and binding kinetics in PSi,^{36,40,41} to investigate porous silicon (PSi) on paper as a new, highly sensitive, quantitative, and reliable platform for RDTs (FIG. 3.3). PSi optical biosensors are well-positioned to have a positive impact on rapid diagnostic technology if further advances are made on the science and engineering related to integrating PSi into a portable, small form factor diagnostic. The key advantages of a future PSi-based rapid diagnostic test are its potential ultra-low detection limit and ability to provide quantification.

PSi-on-paper RDTs would be compatible with both rapid antigen and rapid antibody testing, allowing both direct diagnosis of current infection (antigen) and knowledge of disease progression, disease transmission, and development of herd immunity (antibody). To demonstrate the viability and key advantages of a PSi-on-paper rapid diagnostic test, we first explored techniques to robustly transfer free-standing PSi thin films to paper substrates to enable a cost-effective lateral flow sensor platform with simple analyte acquisition, like traditional platforms used for other RDTs. At the same time, we studied molecular kinetics, binding affinity, and fluid flow dynamics in this new platform through simulations to inform which pore size, morphology, and characteristics provide the best combination of sensitivity, robustness, and response time. Second, we investigated the applicability of the paper-based PSi device to optical biosensing using a well-characterized assay model system with high affinity binding (biotin-streptavidin). The demonstration of a PSi-on-paper sensor capable of reliably and sensitively detecting and quantifying proteins would open the door to a cost-effective and widely deployable RDT platform that could be adapted for a variety of infectious diseases. Such RDTs would provide key information to help healthcare providers make educated treatment and isolation decisions and facilitate epidemiological studies of disease distribution patterns. Moreover, understanding transport and binding kinetics of species inside nanoscale porous materials is essential not only for advancing the capabilities of porous biosensors but also for battery and sorption applications that utilize porous materials.

### Materials

Single-sided, polished, boron-doped, p-type silicon wafers ( [symbol] 100 [symbol] ), 0.01-0.02 Ωcm, 500-550 μm) were purchased from Pure Wafer, Inc. Hydrofluoric acid (HF) (48-51% solution in water) was purchased from Acros Organics. 0.45 μm (47 mm) mixed cellulose ester (MCE) membranes were purchased from Millipore Sigma. For the absorbent pad Cytiva Whatman grade GB003 paper was purchased from Thermo Fisher. For wax printed layers, Whatman grade 4 paper was used. 3-aminopropyl tricthoxysilane (3-APTES), sulfo-NHS-biotin and streptavidin were all purchased from Thermo Fisher. Wax paper printing was done with Xerox ColorQube 8570 printer.

### Porous Silicon Membrane Fabrication

The initial PSi-on-paper system design is shown in FIG. 3.4. In this configuration, PSi sits on a nitrocellulose membrane support, which in turn is contacted with an absorbent pad below, with a Kimwipe in between to facilitate contact. The analyte sample flows vertically through the PSi film and paper to the absorbent pad; capillary forces drive the analyte flow. When the analyte is present in the PSi film, the effective refractive index of the film increases, and the characteristic Fabry-Perot reflectance fringes shift to longer wavelengths.

### PSi Single Layer Design & Fabrication

The first step of realizing the PSi on paper test strip is the fabrication of a PSi free standing membrane. First, a single layer PSi. P-type Si wafer is electrochemically etched at 40 mA/cm² for 635 s, to obtain a single layer PSi of ˜15 μm in thickness. Thin films are difficult to handle, therefore the initial design employed a thicker than usual PSi single layer compared to on substrate PSi optical sensors. It is worth noting that experimental implementation can yield some variation and that the etch calibration curve is only used as a guide for etch condition selection.

### PSi Membrane Lift-off

To detach the layer from the silicon substrate, high current density pulses were applied while the PSi sample remained in the etch cell: 500 mA/cm² twice for 1.7 s separated by 1.7 s intervals, followed by 6 pulses at 420 mA/cm² for 1.5 s each separated by 6 s intervals. The PSi is lifted-off when it appears bulged up inside the etch cell. If not, more pulses at 420 mA/cm² were applied as needed. The slightly detached PSi layer (still attached around the edges) was gently washed with ethanol using a pipettor. The etch cell was disassembled and the PSi on Si substrate removed then introduced into a petri dish filled with ethanol. Gentle turbulence in the ethanol solution were generated close to the PSi sample using a pipettor to help detach the membrane completely. The PSi free-standing membrane (FSM) can then be transferred to another substrate (i.e., glass, paper) either by using tweezers or, if not possible, by using a pipettor and creating light waves in the solution to move it on top of the new substrate. In this case, the liquid is slowly removed from the Petri dish and the PSi membrane is simply picked up while still on the new substrate and allowed to dry in ambient conditions.

### PSi Membrane Oxidation

One of the challenges of using PSi for biosensing applications is that its hydrogen-terminated surface is highly susceptible to corrosion in aqueous media.⁴² In addition to needing surface passivation, as-etched, hydride-terminated porous silicon has a high contact angle and poor wetting properties, which inhibits transport in aqueous media and may block the flow of solutions through the PSi membrane onto the paper support. In order to decrease the water contact angle, passivate the surface against corrosion, and prepare the surface for further chemical modification steps, PSi free-standing membranes were thermally oxidized at 500° C. Here, we note that PSi on silicon is usually oxidized at a higher temperature, such as 800° C. However, employing such high temperature for a thin free-standing membrane may cause damage and therefore a lower oxidation temperature was chosen. The sample (PSi membrane on a supporting substrate) was inserted in the oven at room temperature. The temperature profile was setup to increase slowly at a 5° C./min (over 100 minutes) and the oxidation temperature was maintained for a fixed period (5-600 min) followed by a programmed slow cooling ˜1-2° C./min. The sample was removed from the oven after the temperature returned to room temperature.

### PSi Membrane Characterization

To measure the thickness and pore size of the PSi film, scanning electron microscope (SEM) images were taken of the PSi free-standing membranes. After oxidation, PSi membranes were characterized by ATR-FTIR spectroscopy for determining chemical functional groups on the surface. In addition, Raman spectroscopy measurements were conducted to assess the mechanical properties of PSi membranes. This is achieved by calculating the Raman shift of the silicon peak at ˜520 cm^{˜1.} A shift to a lower wavenumber is indicative of tensile stress experienced by the PSi membrane, while a shift to a higher wavenumber results from compressive stress.⁴³

### PSi Free-Standing Membrane

Etching of PSi single layer resulted in PSi film of ˜15.5 μm with an average pore diameter of 33 nm, as estimated by SEM imaging (FIG. 3.5) and subsequent MATLAB image analysis. After thermal oxidation, the PSi membrane was visibly darker and slightly bent around the edges. This is likely due to the residual stress gained during the etch process, usually compensated for by the silicon substrate. Once the PSi layer is detached, the stress is released in the form of defects/damages in the membrane. Guider et al. studied the mechanical properties of PSi free standing membranes and concluded that both porosity and thickness are correlated to the robustness of the PSi membrane and risk for failure.⁴³ Higher porosity leads to a more fragile structure, indicated by a lower elastic modulus, and thicker films show more residual stress that can cause film damage due to longer etch durations (longer exposure to stress). Hence, the need to optimize the PSi membrane design for an improved mechanical integrity, while preserving its optical properties. If the film is too thick or porosity is too low, thin film interference fringes may not be resolvable. The ATR-FTIR absorbance spectrum of a PSi free-standing membrane oxidized at 500° C. for 5 minutes (FIG. 3.6) shows a strong peak near 1100 cm⁻¹, which corresponds to silicon oxide (Si-Oₓ). In addition, a silicon hydride (OSi-Hₓ) peak is present near 2250 cm⁻¹, indicating that the membrane's is only partially oxidized. As a comparison, unoxidized PSi shows sharp peaks near 2100 cm⁻¹ which correspond to silicon hydride (Si-Hₓ). The Si-Hx peaks are not present after partial oxidation of the PSi FSM.

### PSi-Based Paper Test

Based on PSi free-standing membrane structural and chemical characterization, a thickness of 50 μm and pore size of 33 nm were selected to provide mechanical stability and case of handling/membrane transfer. After fabrication, the oxidized PSi membrane is incorporated into a paper-based support to drive fluid flow through the pores and maximize interactions between analytes and the surface. The following sections describe the assembly and optical signal monitoring in PSi-based paper test.

### Paper Test Assembly

The multiple steps of the realization of the PSi-on-paper sensor are shown in FIG. 3.7. The first step is the fabrication of the PSi membrane. Electrochemical etching in a hydrofluoric acid-based electrolyte was used to form a 50 μm, 33 nm pore diameter PSi film on the silicon substrate (FIG. 3.7a). Next, the PSi film was removed from the substrate by applying a few short, high current density pulses while the PSi sample remained in the electrochemical etching setup. In this way, the bottom part of the PSi film was electropolished and could be separated from the silicon substrate (FIG. 3.7b). The PSi membrane was gradually oxidized at 500° C. in an air ambient for 10 h to increase its surface hydrophilicity (FIG. 3.7c). Next, the PSi was transferred to a nitrocellulose (NC) membrane via wet transfer in water. As shown in FIG. 3.7d-f, the hydrophilic NC membrane (450 nm pore diameter) was inserted in the bottom of a water filled Petri dish. The PSi membrane was added to the Petri dish, and then the water was slowly removed with a pipettor until the PSi touched and formed a good contact with the NC membrane. The PSi-NC system was allowed to dry in air on a paper towel.

The good adhesion between the two membranes is primarily attributed to electrostatic interactions. While the images in FIG. 3.7d-f show PSi membrane attachment to an unaltered NC membrane, to help direct fluid flow in the PSi-on-paper system, we found that it was beneficial to attach the PSi membrane to a wax-printed NC membrane. The wax-printed NC membrane was patterned with a small circular opening where the PSi membrane was electrostatically attached (FIG. 3.7g). Next, wax-printed paper patterned with the same small circular opening was attached as a top layer to the PSi/wax-printed NC membrane system using double-sided tape (FIG. 3.7h). An absorbent pad was then attached to the bottom of the wax-printed NC membrane using double-sided tape. Finally, to ensure that analyte does not leak through the bottom of the absorbent pad, fully filled wax-printed paper was attached to the backside of the absorbent pad as the final layer of the PSi-on-paper system (FIG. 3.7i). Once the paper tested was fabricated it was sandwiched between two plates to achieve a final embodiment of the PSi on paper device (FIG. 3.8). Optical characterization of PSi-on-paper test strip

After assembly of the PSi-on-paper system, reflectance measurements were conducted using a Newport Oriel 6000 Q Series lamp and fiber-coupled Ocean Optics USB-4000 spectrometer. Typical reflectance fringes measured from the PSi membrane are shown in FIG. 3.9. The paper support is sufficiently thick (0.8 mm) such that the measured Fabry-Perot fringes can be attributed entirely to thin film interference from light reflecting off the top and bottom interfaces of the PSi membrane. Consequently, when used for biosensing, changes in the reflectance can be attributed to molecular attachment in the PSi membrane and can be quantified in terms of the number or concentration of molecules attached. The experimental reflectance spectrum and corresponding optical thickness (2nL) were compared to simulated spectrum and 2 nL for film that has a thickness of 50 μm and 67.3% porosity. There is good agreement between experimental and simulated optical properties of the film with slight variation in 2 nL that may be to pore size distribution created during electrochemical etch.

### PSi on Paper Optical Biosensing

### Surface Bio-functionalization

To test the usefulness of the fabricated test in biosensing, a model biotin-streptavidin PSi on paper assay was developed. Surface modification starts after oxidation of PSi free-standing membrane, by submerging it in a 4%(3-aminopropyl)tricthoxysilane solution for 10 min in a glass Petri dish. The PSi membrane is then manually transferred to methanol filled petri dish and soaked for 15 min to remove unreacted APTES. The washed membrane was then removed from methanol and dried on paper towel. Next, the PSi membrane is annealed in the oven at 150° C. for 15 min, to allow cross-linking. To immobilize biotin on the surface, the PSi film is reacted with sulfo-NHS-biotin for 1h in Petri dish containing 0.01 mg/mL sulfo-NHS-biotin in PBS (pH=7.4). Finally, the sample is soaked in water and ethanol and dried on an absorbent pad. FIG. 3.10 shows a schematic of the surface functionalization and the corresponding FTIR spectra. Primary amine peak appears near 1619 cm⁻¹ after silanization reaction, confirming APTES presence. Biotin characteristic peaks near 1710-1640 cm⁻¹ and 3200 cm⁻¹ are also present after NHS-coupling step. Once biotin immobilization into the PSi membrane pores was confirmed, PSi on paper test was assembled as shown in FIG. 3.7e-i, following previously developed protocol.

### Streptavidin PSi on Paper Test

Streptavidin sensing was then performed in real-time by exposing the biotin-functionalized PSi on paper test to a 50 μL solution of streptavidin (80 μM) and continuously recording the reflectance spectrum. A ˜ 35 nm.RIU increase in the optical thickness shown in FIG. 3.11, is observed as the streptavidin solution passed through the PSi measurement window and diminishes only slightly after PBS buffer wash step. The overall redshift indicates the capture of the target protein inside the pores and serves as a first ever demonstration of optical biosensing using PSi on paper.

### Sensor Sensitivity Optimization

To improve the sensitivity of the developed test at practical testing conditions, future optimization should consider structural changes, such as using thinner membrane (FIG. 3.12), without compromising mechanical stability through fabrication and surface modification process. Secondly, the PSi window in the PSi on paper test can be redesigned in the test cassette to accommodate the total sample volume needed without overflowing and causing optical signal loss/disturbances. In all future design improvement strategies, simulations can be used to guide the choice of design parameters paired with experimental validation.

### Summary

The emergence and spread of new infectious diseases have underscored the need to develop highly sensitive, accurate, and cost-effective RDTs that can be widely distributed around the world. The PSi-on-paper optical biosensor platform developed and studied in this work could be applied for the detection of many different infectious diseases, given potential sensitivity enhancement with future design optimization guided with the developed COMSOL models. Whether for a SARS-related virus, influenza, or one of the many insect-borne viral diseases (e.g., malaria, chikungunya, dengue), reliable and highly sensitive RDTs can provide key information to help healthcare providers make educated treatment and isolation decisions and facilitate epidemiological studies of disease distribution patterns. Unlocking an approach that enables high sensitivity and accuracy while maintaining ease of use and low cost would be a tremendous advance in point-of-care diagnostics. Beyond disease detection, understanding transport and binding kinetics of species inside porous silicon is essential for advancing other applications that uses porous materials such as battery and sorption.

### REFERENCES FOR EXAMPLE 3

[1] Caliendo, A. M.; Gilbert, D. N.; Ginocchio, C. C.; Hanson, K. E.; May, L.; Quinn, T. C.; Tenover, F. C.; Alland, D.; Blaschke, A. J.; Bonomo, R. A.; Carroll, K. C.; Ferraro, M. J.; Hirschhorn, L. R.; Joseph, W. P.; Karchmer, T.; MacIntyre, A. T.; Reller, L. B.; Jackson, A. F. Better Tests, Better Care: Improved Diagnostics for Infectious Diseases. Clin. Infect. Dis. 2013, 57 (suppl 3), S139-S170. https://doi.org/10.1093/cid/cit578.
[2] Park, G.-S.; Ku, K.; Back, S.-H.; Kim, S.-J.; Kim, S. Il; Kim, B.-T.; Maeng, J.-S. Development of Reverse Transcription Loop-Mediated Isothermal Amplification Assays Targeting SARS-COV-2. J. Mol. Diagnostics 2020, 22 (6), 1-7.
[3] Pascarella, G.; Strumia, A.; Piliego, C.; Bruno, F.; Del Buono, R.; Costa, F.; Scarlata, S.; Agrò, F. E. COVID-19 Diagnosis and Management: A Comprehensive Review. J. Intern. Med. 2020, Early View (Early View article), 13091. https://doi.org/https://doi.org/10.1111/joim.13091.
[4] US Food and Drug Administration. Coronavirus (COVID-19) Update: FDA Authorizes First Antigen Test to Help in the Rapid Detection of the Virus That Causes COVID-19 in Patients. 2020 [Internet Publication]. 2020, 1-2. https://doi.org/https://www.fda.gov/news-events/press-announcements/coronavirus-covid-19-update-fda-authorizes-first-antigen-test-help-rapid-detection-virus-causes.
[5] Segondy, M. Interests and Limitations of Rapid Diagnostic Tests for Respiratory and Gastrointestinal Viral Diseases. Rev. Francoph. des Lab. 2015, 474, 45-50. https://doi.org/https://doi.org/10.1016/S1773-035X(15)30200-8.
[6] Hu, J.; Wang, S. Q.; Wang, L.; Li, F.; Pingguan-Murphy, B.; Lu, T. J.; Xu, F. Advances in Paper-Based Point-of-Care Diagnostics. Biosens. Bioelectron. 2014, 54, 585-597. https://doi.org/10.1016/j.bios.2013.10.075.
[7] Luo, R.; Fongwen, N.; Kelly-Cirino, C.; Harris, E.; Wilder-Smith, A.; Pecling, R. W. Rapid Diagnostic Tests for Determining Dengue Serostatus: A Systematic Review and Key Informant Interviews. Clin. Microbiol. Infect. 2019, 25 (6), 659-666. https://doi.org/10.1016/j.cmi.2019.01.002.
[8] Jang, I. K.; Tyler, A.; Lyman, C.; Kahn, M.; Kalnoky, M.; Rek, J. C.; Arinaitwe, E.; Adrama, H.; Murphy, M.; Imwong, M.; Ling, C. L.; Proux, S.; Haohankhunnatham, W.; Rist, M.; Seilie, A. M.; Hanron, A.; Daza, G.; Chang, M.; Das, S.; Barney, R.; Rashid, A.; Landier, J.; Boyle, D. S.; Murphy, S. C.; McCarthy, J. S.; Nosten, F.; Greenhouse, B.; Domingo, G. J. Simultaneous Quantification of Plasmodium Antigens and Host Factor C-Reactive Protein in Asymptomatic Individuals with Confirmed Malaria by Use of a Novel Multiplex Immunoassay. J. Clin. Microbiol. 2019, 57 (1), e00948-18.
[9] Xie, J. W.; He, Y.; Zheng, Y. W.; Wang, M.; Lin, Y.; Lin, L. R. Diagnostic Accuracy of Rapid Antigen Test for SARS-COV-2: A Systematic Review and Meta-analysis of 166,943 Suspected COVID-19 Patients. Microbiol. Res. 2022, 265. https://doi.org/10.1016/j.micres.2022.127185.
[10] Tricou, V.; Vu, H. T. T.; Quynh, N. V. N.; Nguyen, C. V. V.; Tran, H. T.; Farrar, J.; Wills, B.; Simmons, C. P. Comparison of Two Dengue NS1 Rapid Tests for Sensitivity, Specificity and Relationship to Viraemia and Antibody Responses. BMC Infect. Dis. 2010, https://doi.org/10.1186/1471-2334-10-142.
[11] Pal, S.; Dauner, A. L.; Mitra, I.; Forshey, B. M.; Garcia, P.; Morrison, A. C.; Halsey, E. S.; Kochel, T. J.; Wu, S. J. L. Evaluation of Dengue Nsl Antigen Rapid Tests and Elisa Kits Using Clinical Samples. PLOS One 2014, 9 (11).

https://doi.org/10.1371/journal.pone.0113411.
[12] Ince, B.; Sezgintürk, M. K. Lateral Flow Assays for Viruses Diagnosis: Up-to-Date Technology and Future Prospects. TrAC-Trends Anal. Chem.2022, 157, 116725. https://doi.org/10.1016/j.trac.2022.116725.
[13] Kosack, C. S.; Page, A. L.; Klatser, P. R. A Guide to Aid the Selection of Diagnostic Tests.Bull. World Health Organ. 2017, 95 (9), 639-645. https://doi.org/10.2471/BLT.16.187468.
[14] Alfthan, H.; Björses, U. M.; Tiitinen, A.; Stenman, U. H. Specificity and Detection Limit of Ten Pregnancy Tests. Scand. J. Clin. Lab. Invest. 1993, 53 (s216), 105-113. https://doi.org/10.1080/00365519309086911.
[15] Bamrungsap, S.; Apiwat, C.; Chantima, W.; Dharakul, T.; Wiriyachaiporn, N. Rapid and Sensitive Lateral Flow Immunoassay for Influenza Antigen Using Fluorescently-Doped Silica Nanoparticles. Microchim. Acta 2014, 181 (1-2), 223-230. https://doi.org/10.1007/s00604-013-1106-4.
[16] Racisossadati, M. J.; Danesh, N. M.; Borna, F.; Gholamzad, M.; Ramezani, M.; Abnous, K.; Taghdisi, S. M. Lateral Flow Based Immunobiosensors for Detection of Food Contaminants. Biosens. Bioelectron. 2016, 86, 235-246. https://doi.org/10.1016/j.bios.2016.06.061.
[17] Akyazi, T.; Basabe-Desmonts, L.; Benito-Lopez, F. Review on Microfluidic Paper-Based Analytical Devices towards Commercialisation. Anal. Chim. Acta 2018, 1001, 1-17.
[18] Zhao, Y.; Lawrie, J. L.; Beavers, K. R.; Laibinis, P. E.; Weiss, S. M. Effect of DNA-Induced Corrosion on Passivated Porous Silicon Biosensors. ACS Appl. Mater. Interfaces 2014, 6 (16), 13510-13519. https://doi.org/10.1021/am502582s.
[19] De Stefano, L.; Rotiroti, L.; Rea, I.; Moretti, L.; Di Francia, G.; Massera, E.; Lamberti, A.; Arcari, P.; Sanges, C.; Rendina, I. Porous Silicon-Based Optical Biochips. J. Opt. A Pure Appl. Opt. 2006, 8, S540.
[20] Rong, G.; Ryckman, J. D.; Mernaugh, R. L.; Weiss, S. M. Label-Free Porous Silicon Membrane Waveguide for DNA Sensing. Appl. Phys. Lett. 2008, No. 93, 161109.
[21] Layouni, R.; Choudhury, M. H.; Laibinis, P. E.; Weiss, S. M. Thermally Carbonized Porous Silicon for Robust Label-Free DNA Optical Sensing. ACS Appl. Bio Mater. 2019, 3 (1), 622-627. https://doi.org/10.1021/acsabm.9b01002.
[22] Rendina, I.; Rea, I.; Rotiroti, L.; De Stefano, L. Porous Silicon-Based Optical Biosensors and Biochips. Phys. E Low-Dimensional Syst. Nanostructures 2007, 38, 188-192.
[23] Dancil, K. P. S.; Greiner, D. P.; Sailor, M. J. A Porous Silicon Optical Biosensor: Detection of Reversible Binding of IgG to a Protein A-Modified Surface. J. Am. Chem. Soc. 1999, 121 (34), 7925-7930.
[24] Pacholski, C.; Sartor, M.; Sailor, M. J.; Cunin, F.; Miskelly, G. M. Biosensing Using Porous Silicon Double-Layer Interferometers: Reflective Interferometric Fourier Transform Spectroscopy. J. Am. Chem. Soc. 2005, 127, 11636-11645. https://doi.org/10.1021/ja0511671.
[25] Bonanno, L. M.; Kwong, T. C.; DeLouise, L. A. Label-Free Porous Silicon Immunosensor for Broad Detection of Opiates in a Blind Clinical Study and Results Comparison to Commercial Analytical Chemistry Techniques. Anal. Chem. 2010, 82 (23), 9711-9718. https://doi.org/10.1021/ac101804s.
[26] Dhanekar, S.; Jain, S. Porous Silicon Biosensor: Current Status. Biosens. Bioelectron. 2013, No. 41, 54-64.
[27] Arshavsky-Graham, S.; Massad-Ivanir, N.; Segal, E.; Weiss, S. Porous Silicon-Based Photonic Biosensors: Current Status and Emerging Applications. Anal. Chem. 2019, 91 (1), 441-467.
[28] Martin-Palma, R. J. Biomedical Applications of Nanostructured Porous Silicon: A Review. J. Nanophotonics 2010, 4 (1), 042502. https://doi.org/10.1117/1.3496303.
[29] Michael J.Sailor. Porous Silicon in Practice: Preparation, Characterization and Applications, 1st Ed.; Wiley-VCH, 2012.
[30] Sailor, M. J. Chemical Reactivity and Surface Chemistry of Porous Silicon. In Handbook of Porous Silicon; Canham, L., Ed.; Springer, Cham, 2014.
[31] Bonanno, L. M.; DeLouise, L. A. Whole Blood Optical Biosensor. Biosens. Bioelectron. 2007, 23 (3), 444-448. https://doi.org/10.1016/j.bios.2007.05.008.
[32] Tong, W. Y.; Sweetman, M. J.; Marzouk, E. R.; Fraser, C.; Kuchel, T.; Voelcker, N. H. Towards a Subcutaneous Optical Biosensor Based on Thermally Hydrocarbonised Porous Silicon. Biomaterials 2016, 74, 217-230.
[33] Schwartz, M. P.; Yu, C.; Alvarez, S. D.; Migliori, B.; Godin, D.; Chao, L.; Sailor, M. J. Using an Oxidized Porous Silicon Interferometer for Determination of Relative Protein Binding Affinity through Non-Covalent Capture Probe Immobilization. Phys. Status Solidi Appl. Mater. Sci. 2007, 204 (5), 1444-1448. https://doi.org/10.1002/pssa.200674380.
[34] Arshavsky-Graham, S.; Massad-Ivanir, N.; Paratore, F.; Scheper, T.; Bercovici, M.; Segal, E. On Chip Protein Pre-Concentration for Enhancing the Sensitivity of Porous Silicon Biosensors. ACS Sensors 2017, 2 (12), 1767-1773.
[35] Cao, T.; Zhao, Y.; Nattoo, C. A.; Layouni, R.; Weiss, S. M. A Smartphone Biosensor Based on Analysing Structural Colour of Porous Silicon. Analyst 2019, 144 (13), 3942-3948. https://doi.org/10.1039/c9an00022d.
[36] Zhao, Y.; Gaur, G.; Retterer, S. T.; Laibinis, P. E.; Weiss, S. M. Flow-through Porous Silicon Membranes for Real-Time Label-Free Biosensing. Anal. Chem. 2016, 88 (22), 10940-10948.
[37] Cao, T.; Zhao, Y.; Nattoo, C. A.; Layouni, R.; Weiss, S. M. A Smartphone Biosensor Based on Analysing Structural Colour of Porous Silicon. Analyst 2019, 144 (13), 3942-3948.
[38] Gallegos, D.; Long, K. D.; Yu, H.; Clark, P. P.; Lin, Y.; George, S.; Nath, P.; Cunningham, B. T. Label-Free Biodetection Using a Smartphone. Lab Chip 2013, 13 (11), 2124-2132. https://doi.org/10.1039/c3lc40991k.
[39] Woodburn, E. V.; Long, K. D.; Cunningham, B. T. Analysis of Paper-Based Colorimetric Assays with a Smartphone Spectrometer. IEEE Sens. J. 2019, 19 (2), 508-514. https://doi.org/10.1109/JSEN.2018.2876631.
[40] Wei, X.; Mares, J. W.; Gao, Y.; Li, D.; Weiss, S. M. Biomolecule Kinetics Measurements in Flow Cell Integrated Porous Silicon Waveguides. Biomed. Opt. Express 2012, 3 (9), 1993.
[41] Zhao, Y.; Gaur, G.; Mernaugh, R. L.; Laibinis, P. E.; Weiss, S. M. Comparative Kinetic Analysis of Closed-Ended and Open-Ended Porous Sensors. Nanoscale Res. Lett. 2016, 11 (1), 395. https://doi.org/10.1186/s11671-016-1614-3.
[42] Jane, A.; Dronov, R.; Hodges, A.; Voelcker, N. H. Porous Silicon Biosensors on the Advance. Trends Biotechnol. 2009, 27 (4), 230-239.
[43] Guider, R.; Traversa, C.; Bettotti, P. Mechanical Stress Relief in Porous Silicon Free Standing Membranes. 2015, 5 (10), 1506-1511. https://doi.org/10.1364/OME.5.002128.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to significant figures of the numerical value. In addition, the phrase “about ‘x’ to ‘y’” includes “about ‘x’ to about ‘y’”.

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

1. A sensor, comprising:
a top cover having at least one opening through the top cover, wherein the top cover has a first side and a second side opposite the first side;
a porous membrane disposed on a first side of a membrane support, wherein the porous membrane has a first side and a second side opposite the first side of the porous support, wherein the porous membrane includes a plurality of pores that extend from the first side of the porous membrane to the second side of the porous membrane, wherein the membrane support has a second side opposite the first side of the membrane support, wherein the second side of the top cover is disposed on the first side of the membrane support;
an absorbent layer having a first side and a second side opposite the first side, wherein the second side of the membrane support is disposed on the first side of the absorbent layer, wherein the absorbent layer has the characteristic of being able to absorb a fluid; and
a bottom cover having a first side and a second side opposite the first side, wherein the second side of the absorbent layer is disposed on the first side of the bottom cover.

2. The sensor of claim 1, wherein the porous membrane is aligned with the opening in the top cover so that the porous membrane is accessible from the first side of the top cover so that a sample fluid is able to contact the porous membrane through the opening of the top cover.

3. The sensor of claim 1, wherein the porous membrane is not aligned with the opening in the top cover; and
further comprising a pathway from the opening in the top cover to the porous membrane, wherein the fluid flows through the pathway from the opening in the top cover to the porous membrane.

4. The sensor of claim 1, wherein the porous membrane is selected from a porous silicon membrane, a porous alumina membrane, a porous silicon dioxide membrane, a porous germanium membrane, a porous germania membrane, or a porous indium phosphide membrane.

6. The sensor of claim 1, wherein the porous membrane has a thickness of about 0.5 to 50 μm.

7. The sensor of claim 1, wherein each of the pores of the plurality of pores independently has a diameter of about 1 to 100 nm.

8. The sensor of claim 1, wherein the plurality of pores has about the same diameter from the first side of the porous membrane to the second side of the porous membrane.

9. The sensor of claim 1, wherein the porous membrane is a stratified porous membrane, wherein the stratified porous membrane has at least two strata and each stratum have a plurality of pores, wherein the pores of one stratum as compared to other strata have a diameter that is different by at least 10 nm.

10. The sensor of claim 9, wherein the stratified porous membrane has a first stratum, a second stratum, and a third strata, wherein the first stratum is on the first side of the porous membrane and the third stratum is on the second side of the porous membrane, wherein the second stratum is positioned between the first stratum and the third stratum, wherein the first stratum has a first plurality of pores, the second stratum has a second plurality of pores, and the third stratum has a third plurality of pores, wherein the first plurality of pores and the third plurality of pores have about the same diameter, wherein the second plurality of pores have a smaller diameter than the first plurality of pore and the third plurality of pores.

11. The sensor of claim 10, wherein the first stratum and the third stratum have a thickness of about 0.5 to 3 μm and the second stratum has a thickness of about 0.5 to 50 μm.

12. The sensor of claim 10, wherein the first plurality of pores have a diameter of about 50 to 80 nm, wherein the third plurality of pores have a diameter of about 50 to 80 nm, wherein the diameter of the pores in the first plurality of pores and the third plurality of pores are the same, wherein the second plurality of pores have a diameter of about 20 to 40 nm.

13. The sensor of claim 1, wherein the porous membrane includes at least one type of binding agent, wherein each type of binding agent has an affinity for a different type of target.

14. The sensor of claim 13, wherein the binding agent is bonded to the surface of the pores of the porous membrane.

15. The sensor of claim 1, wherein the membrane support has a thickness of about 0.5 to 1.5 millimeters, and wherein the absorbent layer has a thickness of about 0.5 to 1.5 millimeters.

16. A sensor, comprising:
a porous membrane disposed on a first side of a membrane support, wherein the porous membrane has a first side and a second side opposite the first side of the porous support, wherein the porous membrane includes a plurality of pores that extend from the first side of the porous membrane to the second side of the porous membrane, wherein the membrane support has a second side opposite the first side of the membrane support; and
an absorbent layer having a first side and a second side opposite the first side, wherein the second side of the membrane support is disposed on the first side of the absorbent layer, wherein the absorbent layer has the characteristic of being able to absorb a fluid.

17. A method of detecting a target, comprising:
exposing a sample fluid on the porous membrane of the sensor of claim 1, wherein if the sample fluid includes the target, the target will be associated with the binding agent and produce a color change in the porous membrane, wherein if the sample fluid does not include the target, the color change does not occur; and
determining if a color change occurs, and if a color change occurs, the target is present in the sample fluid.

18. The method of claim 17, wherein determining includes quantification of the amount of target in the sample fluid.

19. The method of claim 17, wherein the target is selected from the group consisting of:
viruses, archaea, mycoplasma, oligonucleotides, peptides, proteins, enzymes, bacteria, lipids, carbohydrates, metabolites, hormones, antigens, antibodies, and glycoproteins.

20. The method of claim 17, wherein the target is selected from the group consisting of: heavy metals, pyrocatechol, glucose, ochratoxin A, aflatoxin B1, and Fumonisin B1.
